SEQUENCE LISTING (49 sequences)

SEQ ID NO: 1 - DNA, 20 bp, Artificial sequence
  synthetic
gggggacgatcgtcgggggg

SEQ ID NO: 2 - DNA, 20 bp, Artificial sequence
  synthetic
gggggagcatgctgcggggg

SEQ ID NO: 3 - DNA, 24 bp, Artificial sequence
  synthetic
tcgtcgttttgtcgttttgtcgtt

SEQ ID NO: 4 - DNA, 24 bp, Artificial sequence
  synthetic
tgctgcttttgtgcttttgtgctt

SEQ ID NO: 5 - DNA, 20 bp, Artificial sequence
  synthetic
aaaagacgatcgtcaaaaaa

SEQ ID NO: 6 - DNA, 10 bp, Artificial sequence
  synthetic
gggggggggg

SEQ ID NO: 7 - DNA, 10 bp, Artificial sequence
  synthetic
aaaaaaaaaa

SEQ ID NO: 8 - DNA, 10 bp, Artificial sequence
  synthetic
cccccccccc

SEQ ID NO: 9 - DNA, 10 bp, Artificial sequence
  synthetic
tttttttttt

SEQ ID NO: 10 - DNA, 5 bp, Artificial sequence
  synthetic
ggggg

SEQ ID NO: 11 - DNA, 5 bp, Artificial sequence
  synthetic
aagaa

SEQ ID NO: 12 - DNA, 5 bp, Artificial sequence
  synthetic
ttgtt

SEQ ID NO: 13 - DNA, 5 bp, Artificial sequence
  synthetic
ccgcc

SEQ ID NO: 14 - DNA, 5 bp, Artificial sequence
  synthetic
ggggg

SEQ ID NO: 15 - DNA, 5 bp, Artificial sequence
  synthetic
aagga

SEQ ID NO: 16 - DNA, 7 bp, Artificial sequence
  synthetic
ggggggg

SEQ ID NO: 17 - DNA, 5 bp, Artificial sequence
  synthetic
agggg

SEQ ID NO: 18 - DNA, 5 bp, Artificial sequence
  synthetic
aggga

SEQ ID NO: 19 - DNA, 20 bp, Artificial sequence
  synthetic
nntgcatcgatgcagggggg

SEQ ID NO: 20 - DNA, 20 bp, Artificial sequence
  synthetic
nntgcaccggtgcagggggg

SEQ ID NO: 21 - DNA, 20 bp, Artificial sequence
  synthetic
nntgcgtcgacgcagggggg

SEQ ID NO: 22 - DNA, 20 bp, Artificial sequence
  synthetic
nntgcgccggcgcagggggg

SEQ ID NO: 23 - DNA, 20 bp, Artificial sequence
  synthetic
ggtgcatcgatgcagggggg

SEQ ID NO: 24 - DNA, 20 bp, Artificial sequence
  synthetic
ggtgcgtcgacgcagggggg

SEQ ID NO: 25 - DNA, 20 bp, Artificial sequence
  synthetic
ggtgcaccggtgcagggggg

SEQ ID NO: 26 - DNA, 19 bp, Artificial sequence
  synthetic
ggtgcatcgatgcaggggg

SEQ ID NO: 27 - DNA, 24 bp, Artificial sequence
  synthetic
gtttctgtgcacctgtgatgctgt

SEQ ID NO: 28 - DNA, 24 bp, Artificial sequence
  synthetic
actgccagaagtatgggtgagctt

SEQ ID NO: 29 - DNA, 24 bp, Artificial sequence
  synthetic
atttcccacctaccctctggcttt

SEQ ID NO: 30 - DNA, 24 bp, Artificial sequence
  synthetic
tgctctgcatgaggttgtcgatga

SEQ ID NO: 31 - DNA, 23 bp, Artificial sequence
  synthetic
caaggccaaggagctgggagagc

SEQ ID NO: 32 - DNA, 26 bp, Artificial sequence
  synthetic
ggcagaagttccggttatagaagtgc

SEQ ID NO: 33 - DNA, 19 bp, Artificial sequence
  synthetic
gcagcaatggttgacatta

SEQ ID NO: 34 - DNA, 19 bp, Artificial sequence
  synthetic
gcaatggttgacattacta

SEQ ID NO: 35 - DNA, 19 bp, Artificial sequence
  synthetic
gcccagacagtcatgacta

SEQ ID NO: 36 - DNA, 19 bp, Artificial sequence
  synthetic
ggcacctgtgtctggtcta

SEQ ID NO: 37 - DNA, 19 bp, Artificial sequence
  synthetic
gggcatcaccacacttgat

SEQ ID NO: 38 - DNA, 19 bp, Artificial sequence
  synthetic
gcctgtctctgttcttgaa

SEQ ID NO: 39 - DNA, 19 bp, Artificial sequence
  synthetic
gccttgaggccaacaacat

SEQ ID NO: 40 - DNA, 20 bp, Artificial sequence
  synthetic
ggtctatcgtgcatctatga

SEQ ID NO: 41 - DNA, 20 bp, Artificial sequence
  synthetic
ggcttctttcatgtctgtta

SEQ ID NO: 42 - DNA, 19 bp, Artificial sequence
  synthetic
ggggtatcagcgtctaata

SEQ ID NO: 43 - DNA, 19 bp, Artificial sequence
  synthetic
ggtggtgcttcaattaata

SEQ ID NO: 44 - DNA, 19 bp, Artificial sequence
  synthetic
gacccaacttcgataccta

SEQ ID NO: 45 - DNA, 20 bp, Artificial sequence
  synthetic
gcaagtccctggtagaatta

SEQ ID NO: 46 - DNA, 20 bp, Artificial sequence
  synthetic
gtcgattccattaagcaata

SEQ ID NO: 47 - DNA, 19 bp, Artificial sequence
  synthetic
ggcaactgttattacaaga

SEQ ID NO: 48 - DNA, 20 bp, Artificial sequence
  synthetic
gccctgcaaatactagatgt

SEQ ID NO: 49 - DNA, 19 bp, Artificial sequence
  synthetic
ggcgagtgccctgcaaata

(19) United States
(12) Patent Application Publication
(10) Pub. No.: US 2009/0209620 A1
(43) Pub. Date: Aug. 20, 2009

(54) Direct Reversal Of The Suppressive Function Of CD4+Regulatory T Cells Via Toll-Like Receptor 8 Signaling

(76) Inventors: Rong-Fu Wang, Houston, TX (US); Guangyon Peng, Houston, TX (US); Yicheng Wang, Houston, TX (US)

Correspondence Address: FULBRIGHT & JAWORSKI, LLP, 1301 MCKINNEY, SUITE 5100, HOUSTON, TX, 77010-3095, US

(21) Appl. No.: 11/816,902

(22) PCT Filed: Mar. 9, 2006
(86) PCT No.: PCT/US2006/008379

§ 371 Date: Sep. 24, 2008

Related U.S. Application Data
(60) Provisional application No. 60/660,028, filed on Mar. 9, 2005.

(51) Int. Cl. A61K 31/7088; C12N 5/06; C12Q 1/02; C40B 30/06

(52) U.S. Cl. 514/44 A; 435/375; 536/24.5; 435/29; 506/10

(57) ABSTRACT

CD4⁺ regulatory T (Treg) cells profoundly suppress host immune responses and thus protect against autoimmune disease while restricting desired immune responses such as antitumor immunity. Synthetic phosphorothioate-protected, guanosine-containing oligonucleotides can directly reverse the suppressive activity of Treg cells without involving dendritic cells. This effect appears to be transduced by signaling through Toll-like receptor (TLR) 8 and engagement of the MyD88 and IRAK4 molecules in Treg cells. Stimulation of Treg cells with natural ligands for human TLR8 recapitulated the effect of the synthetic guanosine-containing oligonucleotides .

### CROSS-REFERENCE TO RELATED APPLICATIONS

This application claims the benefit of Provisional U.S. application 60/660,028, the contents of which are incorporated by reference into this application.

### STATEMENT REGARDING FEDERALLY SPONSORED RESEARCH OR DEVELOPMENT

This invention was made in part with government support under Grant Nos. R01CA90327, R01CA101795, P50CA58204, P50CA093459 and PO1CA94237 awarded by the National Institutes of Health. The United States Government may have certain rights in the invention.

### BACKGROUND OF THE INVENTION

Naturally occurring CD4⁺ CD25⁺ regulatory T cells (Treg cells) and antigen-induced Treg cells induce self-tolerance by suppressing host immune responses, and thus play critical roles in the prevention of many autoimmune diseases. However, Treg cells can have detrimental effect on immunotherapy for cancer or other illnesses because they potently suppress immune responses elicited by vaccination or other systemic antigen stimulation. Increased proportions of CD4⁺ CD25⁺ Treg cells in total CD4⁺ T cell populations have been observed in patients with different types of cancers, including lung, breast and ovarian tumors. Antigen-specific CD4⁺ Treg cells from fresh tumor tissues from patients can be isolated and tumor infiltrating lymphocyte (TIL) lines established. These cells suppressed the proliferation of naive CD4⁺ T cells and inhibited interleukin (IL)-2 secretion by CD4⁺ effector cells through a cell-cell contact mechanism, and their suppressive function could not be reversed by a high concentration of IL-2. Thus, both naturally occurring CD4⁺ CD25⁺ and tumor-specific Treg cells present at tumor sites pose a major obstacle to successful immunotherapy for cancer and other diseases.

Toll-like receptors (TLRs) recognize a set of conserved molecular structures, so called pathogen associated molecular patterns (PAMPs), allowing them to sense and initiate innate and adaptive immune responses. Such responses are generally thought to be produced through the induction of a dendritic cell (DC) maturation program that enables DCs to activate naive T cells and to acquire the potential to control Treg cells. Stimulation of mouse DCs with TLR ligands such as lipopolysaccharide (LPS) and CpG DNA has been reported to induce DCs to secrete cytokines such as IL-6 and to render CD4⁺ effector cells refractory to Treg cell-mediated suppression.

### BRIEF SUMMARY OF THE INVENTION

The invention relates to a method for inhibiting the immunosuppressive capacity of CD4⁺ CD25⁺ Treg cells and antigen-induced Treg cells. The immunosuppressive activity of these cells is down regulated by short guanine containing oligonucleotides through the TLR8-IRKA4-MyD88 signal transduction pathway. The invention also discloses a method for identifying compounds which inhibit the immunosuppressive capacity of CD4⁺ CD25⁺ Treg cells and antigen-induced Treg cells. The method includes a comparison of cellular growth and/or division rates of parallel samples of näive CD4⁺ T cells. Näive CD4⁺ T cells exposed to uninhibited Treg cells are compared to control näive CD4⁺ T cells and näive CD4⁺ T cells exposed to Treg cells treated with a compound. The reversal of Treg suppression is measured by the relative growths of the variously treated näive CD4⁺ T cells. The invention also includes application of identified inhibitory compounds to decrease Treg cell mediated immunosuppression in the context of an organism suffering a disease such as an infection or cancer. The resultant increase in immune activity helps the organism's immune response to combat the disease state.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention. All articles, papers and other references cited herein are incorporated by reference. This incorporation by reference includes the articles, papers and other references listed within or otherwise cited by these incorporated articles, papers and other references.

### BRIEF DESCRIPTION OF THE DRAWINGS

For a more complete understanding of the present invention, reference is now made to the following descriptions taken in conjunction with the accompanying drawing, in which:

FIG. 1. Reversal of the suppressive function of CD4⁺ Treg cells by CpG-A. A. Restoration of the proliferation of naive CD4⁺ T cells suppressed by CD4⁺ Treg cells in an assay system containing DCs stimulated with TLR ligands or cytokines. B. Requirement for DCs in reversing the suppressive function of Treg cells on naive T cell proliferation. C. Pretreatment of CD4⁺ Treg cells with CpG-A (SEQ ID NO: 1) or non-CpG-A (SEQ ID NO: 3) reverses their suppressive function.

FIG. 2. Identification of sequence elements in CpG-A responsible for the direct reversal of the suppressive function of CD4⁺ Treg cells. A. Poly-G, but not the CpG motif, is responsible for the observed reversal effect. B. Poly-A10 (SEQ ID NO: 6), Poly-T10 (SEQ ID NO: 9) and Poly-C10 (SEQ ID NO: 8) failed to reverse the suppressive function of Treg cells. C. Minimal number of guanosine nucleosides required for reversing the suppressive function of Treg cells. D. Ability of A4G1 (SEQ ID NO: 11), T4G1 (SEQ ID NO: 12) and C4G1 (SEQ ID NO: 13) oligonucleotides to reverse the suppressive function of CD4⁺ Treg cells. E. Reversal of suppressive function of naturally occurring CD4⁺ CD25⁺ Treg cells by Poly-G5 (SEQ ID NO: 14). F. A list of oligonucleotide DNA sequences. * stand for phosphorothioate linkage.

FIG. 3. MyD88-IRAK4 pathway is required for reversing the suppressive function of Treg cells. A. Knock down of IRAK4 and MyD88 by RNA interference. B. Purification of Treg cells transduced with IRAK4 siRNAl and MyD88 siRNAl. C. Evaluation of the reversibility of transduced (GFP⁺) and untransduced (GFP⁻) Treg cells by Poly-G10 (SEQ ID NO: 6) oligonucleotides.

FIG. 4. TLR8 is the receptor responsible for Guanine oligonucleotide-induced reversal of the suppressive function of Treg cells. A. Pattern of TLR7, 8 and 9 expression in Treg cells determined by RT-PCR with gene-specific primers. B. TLR7 and 8 expression determined by real-time PCR analysis of cDNA. C. Knock down of TLR7, 8 and 9 by siRNAs. D. Partial loss of reversible suppressive function by Treg cells transduced with TLR8 siRNA. E. Evaluation of various TLR ligands for their ability to reverse the suppressive function of Treg cells. F. Poly-G10 (SEQ ID NO: 6)-induced reversal of Treg cell function enhances antitumor immunity in vivo.

FIG. 5. Proliferation and cytotoxicity of Treg cells.

FIG. 6. Identification and titration of sequence elements in CpG-A responsible for the direct reversal of Treg cell suppressive function.

FIG. 7. Purification and suppressive function of CD4⁺ CD25⁺ Treg cells and their functional reversal by Poly-G5.

FIG. 8. Knockdown of IRAK4, MyD88, TLR7, TLR8 and TLR9 by RNA interference.

FIG. 9. The MyD88-IRAK4 pathway is required to reverse the suppressive function of Treg164 cells.

FIG. 10. Expression level of TLR7 and 8 in Treg cells determined by real-time PCR analysis in different cell lines with gene-specific primers.

### DETAILED DESCRIPTION OF THE INVENTION

Definitions

As used herein, the use of the word “a” or “an” when used in conjunction with the term “comprising” in the claims and/or the specification may mean “one,” but it is also consistent with the meaning of “one or more,” “at least one,” and “one or more than one.” Still further, the terms “having,” “including,” “containing” and “comprising” are interchangeable and one of skill in the art is cognizant that these terms are open ended terms. Some embodiments of the invention may consist of or consist essentially of one or more elements, method steps, and/or methods of the invention. It is contemplated that any method or composition described herein can be implemented with respect to any other method or composition described herein.

Unless defined otherwise, technical and scientific terms used herein have the same meaning as commonly understood by one of ordinary skill in the art to which this invention belongs. For purposes of the present invention, the following terms are defined below.

“An effective amount” is a concentration of oligonucleotide in a Treg cell's environment capable of inhibiting the Treg cell's immunosuppressive activity. The term “therapeutically effective amount” as used herein refers to an amount that results in an improvement or remediation of the symptoms of the disease or condition.

The term “nucleic acid” is well known in the art. A “nucleic acid” as used herein will generally refer to a molecule (i.e., a strand) of DNA, RNA or a derivative or analog thereof, comprising a nucleobase. A nucleobase includes, for example, a naturally occurring purine or pyrimidine base found in DNA (e.g., an adenine “A,” a guanine “G,” a thymine “T” or a cytosine “C”) or RNA (e.g., an A, a G, an uracil “U” or a C). The term “nucleic acid” encompass the terms “oligonucleotide” and “polynucleotide,” each as a subgenus of the term “nucleic acid.” The term “oligonucleotide” refers to a molecule of between about 3 and about 100 nucleobases in length. The term “polynucleotide” refers to at least one molecule of greater than about 100 nucleobases in length. These definitions generally refer to a single-stranded molecule, but in specific embodiments will also encompass an additional strand that is partially, substantially or fully complementary to the single-stranded molecule. Thus, a nucleic acid may encompass a double-stranded molecule or a triple-stranded molecule that comprises one or more complementary strand(s) or “complement(s)” of a particular sequence comprising a molecule. As used herein, a single stranded nucleic acid may be denoted by the prefix “ss,” a double stranded nucleic acid by the prefix “ds,” and a triple stranded nucleic acid by the prefix “ts.” Preferably, in nucleic acids comprising natural organic bases, the bases are unmethylated. Nucleic acid molecules can be obtained from existing nucleic acid sources but are preferably synthetic.

The term “library” includes searchable populations of small molecules or mixtures of molecules. In one embodiment, the library is comprised of samples or test fractions (either mixtures of small molecules or isolated small molecules) which are capable of being screened for activity. For example, the samples could be added to wells in a manner suitable for high throughput screening assays. In a further embodiment, the library could be screened for binding compounds by contacting the library with a target of interest, e.g., a live cell, a protein or a nucleic acid.

“Type D CpG oligonucleotides” are well known in the art as disclosed by U.S. Pat. No. 6,977,245 which is incorporated by reference. Type D CpG oligonucleotides generally contain a CpG dinucleotide sequence and a stretch of 4 or more contiguous guanine residues. Type D CpG oligonucleotides are generally between 18 and 30 nucleotides in length, and may contain one or more of the following sequence content:

| | |
|---|---|
| 5′-X₁X₂TGCATCGATGCAGGGGGG-3′; | (SEQ ID NO:19) |
| 5′-X₁X₂TGCACCGGTGCAGGGGGG-3′; | (SEQ ID NO:20) |
| 5′-X₁X₂TGCGTCGACGCAGGGGGG-3′; | (SEQ ID NO:21) |
| 5′-X₁X₂TGCGCCGGCGCAGGGGGG-3′; | (SEQ ID NO:22) |
| 5′-GGTGCATCGATGCAGGGGGG-3′; | (SEQ ID NO:23) |
| 5′-GGTGCGTCGACGCAGGGGGG-3′; | (SEQ ID NO:24) |
| 5′-GGTGCACCGGTGCAGGGGGG-3′; | (SEQ ID NO:25) |
| or | |
| 5′-GGTGCATCGATGCAGGGGG-3′;, | (SEQ ID NO:26) |

where X may be any nucleobase or none.

A “non CpG containing recombinant DNA” is a recombinant DNA that is does not contain a CpG dinucleotide sequence.

A “nuclease resistant inter-residue backbone linkage” is a chemical linkage between organic bases in a nucleic acid that is more resistant to in vivo nuclease degradation as compared to naturally occurring phosphodiester linkages. The preferred chemical linkage is a phosphorothioate (i.e., at least one of the phosphate oxygens of the nucleic acid molecule is replaced by sulfur) or phosphorodithioate modified nucleic acid molecules. Other stabilized nucleic acid molecules include: nonionic DNA analogs, such as alkyl- and aryl-phosphonates, phosphodiester and alkylphosphotriesters, in which the charged oxygen moiety is alkylated. Nucleic acid molecules which contain a diol, such as tetraethyleneglycol or hexaethyleneglycol, at either or both termini have also been shown to be substantially resistant to nuclease degradation.

A “nuclease sensitive inter-residue backbone linkage” is a phosphodiester linkage between organic bases in a nucleic acid or alternative known in the art which degrades in vivo from nuclease activity at least at the same rate as a phosphodiester linkage.

An “immunogenic composition” is any composition capable of eliciting an immune response in a subject upon administration. The term “vaccine” as used herein is defined as material used to provoke an immune response (e.g., the production of antibodies) on administration of the materials and thus conferring immunity. Thus, a vaccine is an antigenic and/or immunogenic composition.

“Regulatory T cells” (Treg cells) are a functionally defined subset of CD4⁺ T lymphocytes. Treg cells function in vivo to control immunological reactivity to self antigens. This function is manifested by Treg cells' ability to suppress the activation of näive immune effector cells (CD4⁺ and CD8⁺ ) such as CD4⁺ CD25⁻ T cells. Two major classes of Treg cells are the thymically derived natural Treg cells and antigen induced Treg cells. Naturally occurring Treg cells mediate immunotolerance of self-antigens and their dysregulation may play a role in autoimmune diseases. Antigen induced Treg cells are induced by peripheral antigen stimulation. This subcategory of Treg cells is found among tumor infiltrating lymphocytes and mediates tolerance of tumor antigens. While Treg cell activation can be antigen specific, Treg immunosuppression is not. Thus, Treg activity creates a globally suppressive immunological state. Treg cells have been characterized as a subpopulation of CD4⁺ T-cells expressing the IL-2 receptor CD25. However, some experiments demonstrate that CD25 may not be expressed by Treg cells under some conditions. Other molecular markers strongly associated with Treg cells are the transcription factor, FOXP3, and glucocorticoid-induced tumor necrosis factor receptor family-related gene (GITR, also known as TNFRSF18). However, none of these molecular markers are determinate of Treg identity or completely correlate with Treg immunosuppression activity.

“Cytokines” are small secreted proteins which mediate and regulate immunity, inflammation, and hematopoiesis. They must be produced de novo in response to an immune stimulus. They generally (although not always) act over short distances and short time spans and at very low concentration. They act by binding to specific membrane receptors, which then signal the cell via second messengers, often tyrosine kinases, to alter its behavior. Responses to cytokines include increasing or decreasing expression of membrane proteins (including cytokine receptors), proliferation, and secretion of effector molecules. Cytokine is a general name; other names include lymphokine (cytokines made by lymphocytes), monokine (cytokines made by monocytes), chemokine (cytokines with chemotactic activities), and interleukin (cytokines made by one leukocyte and acting on other leukocytes). Cytokines may act on the cells that secrete them (autocrine action), on nearby cells (paracrine action), or in some instances on distant cells (endocrine action). Cytokines are made by many cell populations, but the predominant producers are helper T cells (Th) and macrophages. The largest group of cytokines stimulates immune cell proliferation and differentiation. This group includes Interleukin 1 (IL-1), which activates T cells; IL-2, which stimulates proliferation of antigen-activated T and B cells; IL-4, IL-5, and IL-6, which stimulate proliferation and differentiation of B cells; Interferon gamma (IFNg), which activates macrophages; and IL-3, IL-7 and Granulocyte Monocyte Colony-Stimulating Factor (GM-CSF), which stimulate hematopoiesis.

“Subject” is an organism being given a nucleic acid according to the methods disclosed by the Specification. Preferably a subject expresses a functional TLR8 on the subject's Treg cells. Preferably, a subject is a mammal other than mice (which do not express a functional TLR8), more preferably human.

The practice of the present invention will employ, unless otherwise indicated, conventional techniques of molecular biology, microbiology, recombinant DNA, and so forth which are within the skill of the art. Such techniques are explained fully in the literature. See e.g., Sambrook, Fritsch, and Maniatis, MOLECULAR CLONING: A LABORATORY MANUAL, Second Edition (1989), OLIGONUCLEOTIDE SYNTHESIS (M. J. Gait Ed., 1984), ANIMAL CELL CULTURE (R. I. Freshney, Ed., 1987), the series METHODS IN ENZYMOLOGY (Academic Press, Inc.); GENE TRANSFER VECTORS FOR MAMMALIAN CELLS (J. M. Miller and M. P. Calos eds. 1987), HANDBOOK OF EXPERIMENTAL IMMUNOLOGY, (D. M. Weir and C. C. Blackwell, Eds.), CURRENT PROTOCOLS IN MOLECULAR BIOLOGY (F. M. Ausubel, R. Brent, R. E. Kingston, D. D. Moore, J. G. Siedman, J. A. Smith, and K. Struhl, eds., 1987), CURRENT PROTOCOLS IN IMMUNOLOGY (J. E. Coligan, A. M. Kruisbeek, D. H. Margulies, E. M. Shevach and W. Strober, eds., 1991); ANNUAL REVIEW OF IMMUNOLOGY; as well as monographs in journals such as ADVANCES IN IMMUNOLOGY.

Description

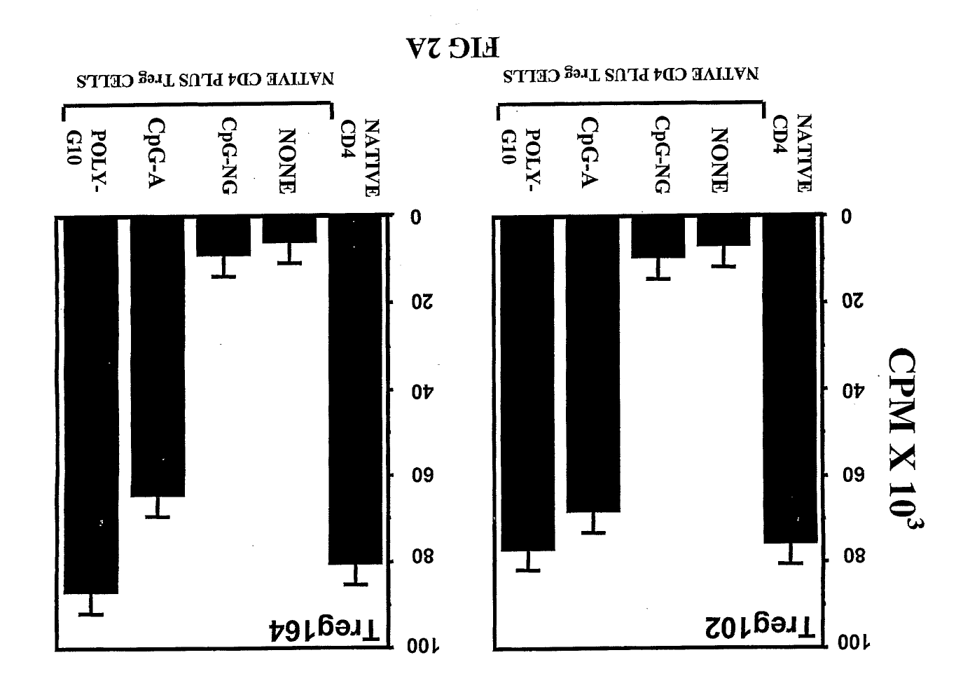

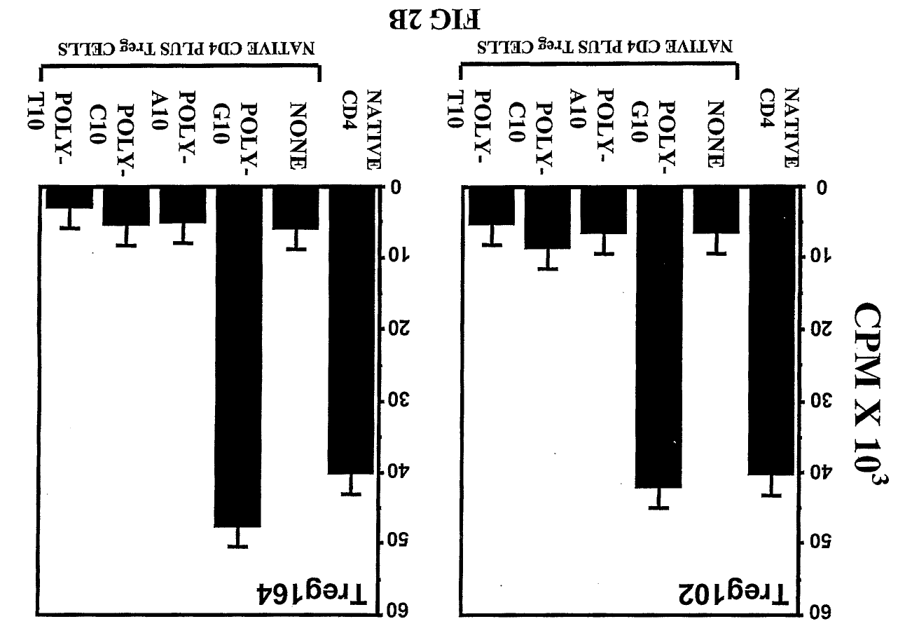

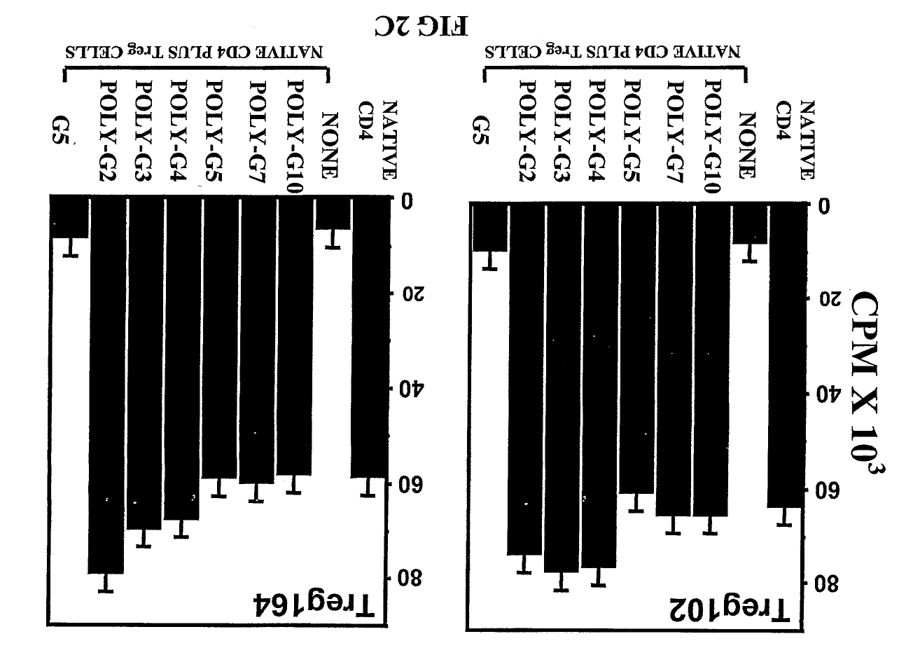

A new class of immunologically active oligonucleotides are disclosed. These immunologically active oligonucleotides act through Toll-Like Receptor 8 (TLR8) to activate the TLR8-IRKA4-MyD88 signal transduction pathway in regulatory T cells (Treg cells). This signaling down regulates Treg cell activity leading to a derepression of immunological activity. In one embodiment, this new class of immunologically active oligonucleotides includes oligonucleotides with a guanosine and a partially stabilized or nuclease resistant inter-residue backbone. A representative group of oligonucleotides is shown in FIG. 2F (* indicates a nuclease resistant inter-residue backbone linkage.). This new class of oligonucleotides excludes CpG-A or Type D CpG oligonucleotides already known in the art. As shown in FIG. 2C, this new class of immunologically active oligonucleotides does not depend on having a CpG dinucleotide sequence. It is preferred that the oligonucleotide be a deoxyribonucleic acid for stability reasons, but other embodiments may include ribonucleic acids. The preferred length of the oligonucleotides is from about 4 to about 15 nucleotides, more preferably about 5 to about 10 nucleotides. In embodiments with partially stabilized backbones, it is preferred to have a nuclease resistant inter-residue linkages between a guanosine and an adjacent residue.

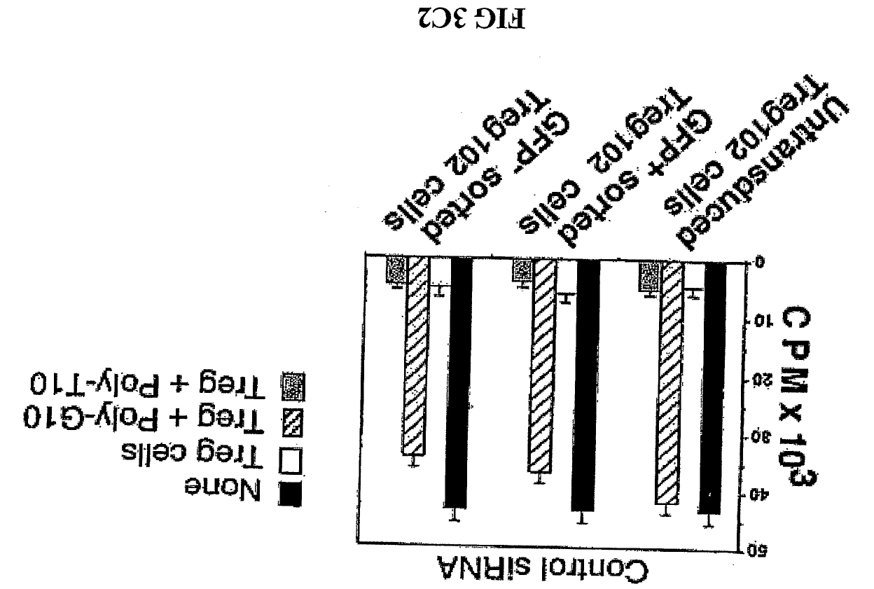

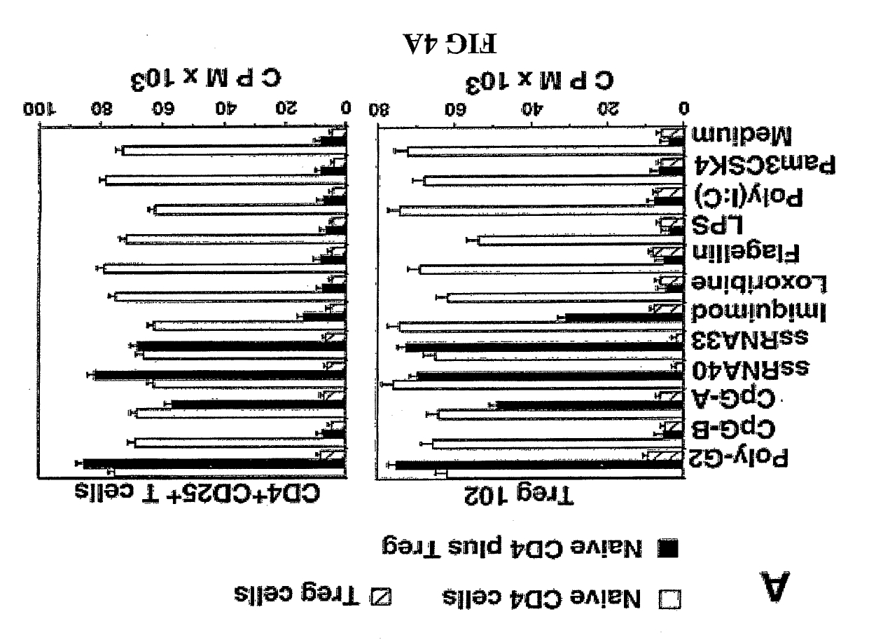

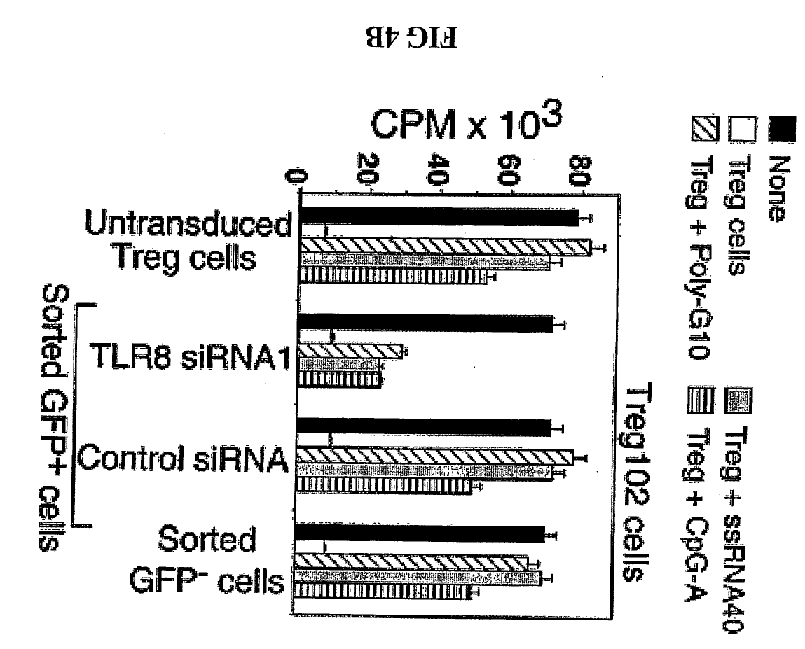

Another embodiment of the invention shown in relates to methods for inhibiting the immunosuppressive capacity of Treg cells utilizing this new class of immunologically active oligonucleotides. As shown in FIG. 2A-E, the immunosuppressive activity of Treg cells against näive CD4⁺ T-cells is down regulated by an effective amount of a guanine containing oligonucleotide. In a particular embodiment, Treg cell activity is down regulated in vitro with the effective amount determined by a titration series of oligonucleotide dosages (FIG. 6). This Treg suppression method is effective with antigen specific Treg cells from tumors (FIG. 2 A-D) and thymically derived circulating Treg cells (FIG. 2E). Therefore, this method of suppressing Treg cell activity may be applied effectively in a wide variety of contexts such as in subjects with an infectious disease or cancer.

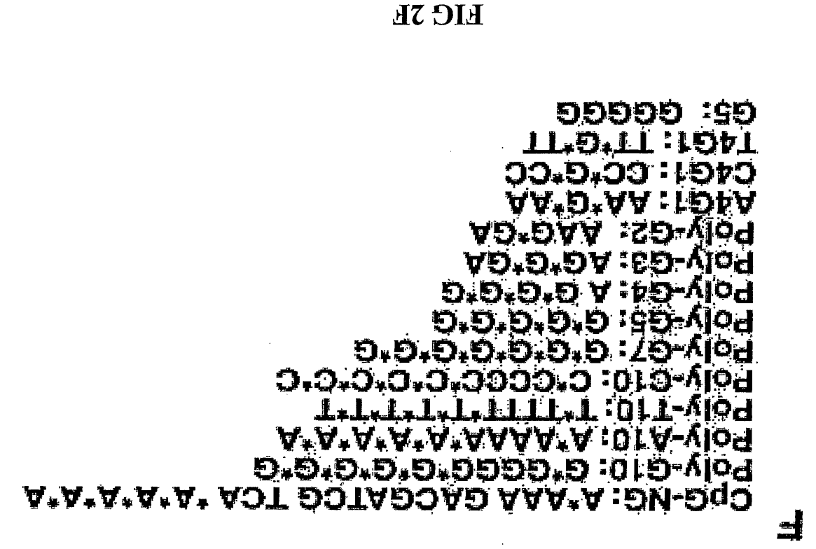

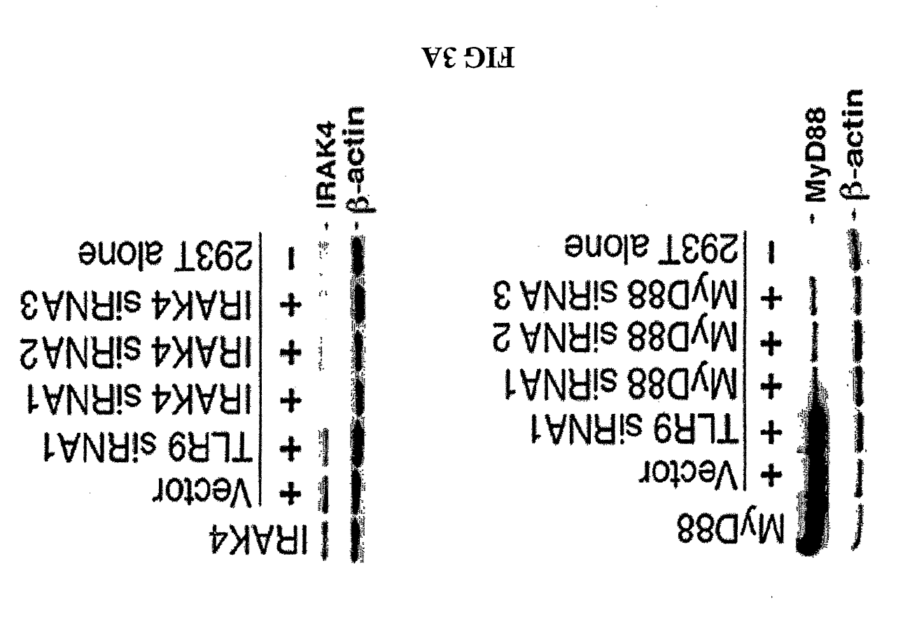

Another embodiment of the invention relates to a new method for identifying compounds which inhibit the immunosuppressive capacity of CD4⁺ CD25⁺ Treg cells and antigen-induced Treg cells. In a preferred embodiment, demonstrated in FIG. 4A, the method includes a comparison of cellular growth and/or division rates of parallel samples of näive CD4⁺ T cells. Näive CD4⁺ T cells exposed to uninhibited Treg cells are compared to 1) control näive CD4⁺ T cells and 2) näive CD4⁺ T cells exposed to Treg cells treated with a compound of interest. The reversal of Treg suppression is measured by the relative growths rates of the variously treated näive CD4⁺ T cells. In a particular embodiment, the method for identifying compounds is used to screen a library or collection of compounds. Such libraries are well known in the art and widely available (e.g., the NIH Molecular Libraries Small Molecule Repository http://mlsmr.discoverypartners.com/MLSMR_HomePage/index.html). Lead compounds identified by a library screen can subsequently be modified to derive pharmaceutically acceptable compounds for reversing immunosuppression by Treg cells. In a preferred embodiment, the method for identifying compounds is semi- or fully automated using robotic systems and other devices well known in the art for high throughput library screening of cell based assays. (See, e.g., U.S. Pat. No. 6,400,487 Method and apparatus for screening chemical compounds).

### EXAMPLES

Establishing Treg cell lines and purifying natural CD4⁺ CD25⁺ Treg cells CD4⁺ Treg clones were established from CD4⁺ tumor-infiltrating lymphocytes (TIL102 and TIL164) and maintained in RPMI 1640 medium containing 10% human AB serum and recombinant IL-2 (300 IU/ml) using methods well known in the art (See, e.g., H. Y. Wang et al., Immunity 20, 107-118 (2004)). Treg clones derived from TIL102 or TIL164 cells were pooled and designated Treg102 or Treg164. Naturally occurring CD4⁺ CD25⁺ Treg cells were obtained by sorting CD4⁺ T cell populations from fresh PBMCs after staining with anti-CD4 and anti-CD25 antibodies. To obtain optimal expansion, the OKT3 expansion method was performed using methods well known in the art (See, e.g., H. Y. Wang et al., Immunity 20, 107-118 (2004)). T cell clones were maintained at a low IL-2 concentration (300 IU/ml). Melanoma cell lines and EBV-transformed B-cell lines used in this study were cultured in RPMI 1640 medium containing 10% FCS. Human embryonic kidney (HEK) 293 and Epstein-Barr virus (EBV)-transformed B cell lines were maintained in RPMI 1640 with 10% fetal calf serum (FCS).

Assays for Treg Suppression of Näive T Cell Proliferation

A. Proliferation assay with Dendritic Cells (DCs): Näive CD4⁺ T cells were purified from PBMCs using microbeads (Miltenvi Biotec). DCs were generated from PBMC-derived monocytes in the presence of IL-4 (500 ng/ml) and GM-CSF (800 ng/ml) for 7 days. 1×105 näive CD4⁺ T cells were cultured with regulatory T cells at different ratios (1: 0.2, 1:0.1 and 1:0.05) in 200 ul of medium containing 2×104 of DCs and anti-CD3 antibody (100 ng/ml) plus one of the following TLR ligands or cytokines: CpG-A (3 ug/ml), CpG-B (3 ug/ml), LPS (100 ng/ml), TNF.-α (20 ng/ml), IFN-α (100 ng/ml), or IL-6 (100 ng/ml). After 56 h of culture, [3H]thymidine was added at a final concentration of 1 uCi/well, followed by an additional 16 h. of culture. The incorporation of [³H]thymidine was measured with a liquid scintillation counter, using methods well known in the art (See, e.g., M. K. Levings et al., J. Exp. Med. 196, 1335-46 (2002)). All experiments were performed in triplicate.

B. Proliferation assay without DCs: 1×105 näive CD4 T cells were cultured with regulatory T cells at different ratios (1: 0.2, 1:0.1 and 1:0.05) in anti-CD3 mAb-coated (2 ug/ml) 96-well plates in the presence or absence of the following TLR ligands or cytokines: CpG-A (3 ug/ml), CpG-B (3 ug/ml), LPS (100 ng/ml), TNF.-α (20 ng/ml), IFN-α (100 ng/ml), or IL-6 (100 ng/ml). After 56 h of culture, [3H]thymidine was added at a final concentration of 1 uCi/well, followed by an additional 16 h of culture. The incorporation of [³H]thymidine was measured with a liquid scintillation counter.

C. Pretreatment of Treg cells: 1×106 Treg cells were cultured in medium containing CpG-A (3 ug/ml), CpG-B (3 ug/ml), LPS (100 ng/ml), TNF.-α (20 ng/ml), IFN-α (100 ng/ml) or IL-6 (100 ng/ml) for 3 days. After three washes, the pretreated Treg cells were cultured with näive CD4⁺ T cells in anti-CD3 mAb-coated plates (without DCs) to determine their suppressive function. After 56 h of culture, [3H]thymidine was added at a final concentration of 1 uCi/well, and cultured for an additional 16 h. The incorporation of [³H]thymidine was measured with a liquid scintillation counter.

FACS Analysis

The expression of CD4, CD25 and GITR on Treg cells was determined by FACS analysis after staining with specific antibodies (purchased from R&D Systems and BD Biosciences). To isolate naturally occurring CD4⁺ CD25⁺ Treg cells, CD4⁺ T cells were stained with anti-CD4 and anti-CD25 antibodies conjugated to either PE or FITC. After washing, the cells were sorted by FACSARIA into CD4⁺ CD25⁺ and CD4⁺ CD25⁻ T cell populations. For experiments with carboxyfluorescein diacetate succinimidyl ester (CFSE)-labeled cells, näive CD4⁺ T cells or Treg cells (1×107) were stained with CFSE (4.5 uM) from Molecular Probes at 37° C. for 15 min. After several washes, the labeled cells were cultured in RPMI 1640 containing 10% human AB serum and IL-2 (300 IU/ml). To determine suppression of cell division by Treg cells, unlabeled Treg cells were added to CFSE-labeled näive T cells at an 1:1 ratio in 24-well plates precoated with OKT3 (2 ug/ml) in the presence or absence of CpG-A or Poly-G2 (3 ug/ml). After 3 days in culture, the cells were analyzed by FACS gating on the CFSE-labeled cells.

rtPCR and Quantitative PCR

Total RNA was extracted from T cells with Trizol reagent (Invitrogen, Inc. San Diego, Calif.) and SuperScript II RT kit, reverse transcription (Invitrogen, Inc. San Diego, Calif.). A 20-μl reverse transcription mixture containing 2 μg of total RNA was incubated at 42° C. for 1 h. TLR7, 8 and 9 mRNA levels were quantified by real-time PCR with ABI/PRISM7000 sequence detection system (PE Applied Biosystems, Inc. Foster City, Calif.). PCR reactions were performed with primers, an internal fluorescent TaqMan probe specific to TLR7, 8, 9, MyD88, IRAK4 or HPRT (purchased from PE Applied Biosystems Inc., Foster City, Calif.). TLR7, 8 and 9, MyD88 and IRAK4 mRNA levels in each sample were normalized to the relative quantity of HPRT. All samples were run in triplicate. For specific knockdown of TLR8, MyD88 and IRAK4 in Treg cells, Treg cells were infected with the corresponding siRNA for each gene and sorted them into transduced (GFP⁺) and untransduced (GFP⁻) cell populations. Total RNA was extracted from both transduced (GFP⁺) and untransduced (GFP⁻) cell populations to determine the expression level of a relevant gene by real-time PCR. The expression level of an irrelevant gene served as a control. For conventional reverse-transcription PCR, gene-specific primers were used and the reactions run for 28 cycles of 94° 2 min for initial denature, 28 cycles of 94° for 30 s, 56° for 30 s and 72° for 45 s, followed by extension at 72° for 10 min. PCR products were separated on 1% agarose gel.

The following primers were used for TLR7

(SEQ ID NO:27)

(TLR7-5P, 5′ GTTTCTGTGCACCTGTGATGCTGT;

(SEQ ID NO:28)

TLR7-3P, 5′ ACTGCCAGAAGTATGGGTGAGCTT),

for TLR8

(SEQ ID NO:29)

(TLR8-5P, 5′-ATTTCCCACCTACCCTCTGGCTTT;

(SEQ ID NO:30)

TLR8-3P, 5′ TGCTCTGCATGAGGTTGTCGATGA),

and for TLR9

(SEQ ID NO:31)

(TLR9-5P, 5′CAAGGCCAAGGAGCTGGGAGAGC;

(SEQ ID NO:32)

TLR9-3P, 5′ GGCAGAAGTTCCGGTTATAGAAGTGC).

Lentivirus-Based siRNAs

Several siRNA sequences (19 nucleotides) for each gene were selected with use of computer-assisted programs. Oligonucleotides containing a siRNA sequence, 8 nucleotide spacers, and a polyT terminator sequence were annealed and then cloned into the HapI and XhoI sites of GFP-expressing pLentilox3.7 vector using methods well known in the art (See, e.g., D. A. Rubinson et al., Nat. Genet. 33, 401-6 (2003)). siRNA was under the control of a U6 promoter. The following DNA sequences used to construct siRNAs for IRAK4, MyD88, and TLR7, 8 and 9, were effective in silencing their corresponding genes:

| | | |
|---|---|---|
| IRAK4 | 5′GCAGCAATGGTTGACATTA; | (SEQ ID NO:33) |
| siRNA1, | | |
| IRAK4 | 5′GCAATGGTTGACATTACTA; | (SEQ ID NO:34) |
| siRNA2, | | |
| IRAK4 | 5′GCCCAGACAGTCATGACTA; | (SEQ ID NO:35) |
| siRNA3, | | |
| MyD88 | 5′GGCACCTGTGTCTGGTCTA; | (SEQ ID NO:36) |
| siRNA1, | | |
| MyD88 | 5′GGGCATCACCACACTTGAT; | (SEQ ID NO:37) |
| siRNA2, | | |
| MyD88 | 5′GCCTGTCTCTGTTCTTGAA; | (SEQ ID NO:38) |
| siRNA3, | | |
| TLR7 | 5′GCCTTGAGGCCAACAACAT; | (SEQ ID NO:39) |
| siRNA1, | | |
| TLR7 | 5′GGTCTATCGTGCATCTATGA; | (SEQ ID NO:40) |
| siRNA2, | | |
| TLR7 | 5′GGCTTCTTTCATGTCTGTTA; | (SEQ ID NO:41) |
| siRNA3, | | |
| TLR7 | 5′GGGGTATCAGCGTCTAATA; | (SEQ ID NO:42) |
| siRNA4, | | |
| TLR8 | 5′GGTGGTGCTTCAATTAATA; | (SEQ ID NO:43) |
| siRNA1, | | |
| TLR8 | 5′GACCCAACTTCGATACCTA; | (SEQ ID NO:44) |
| siRNA2, | | |
| TLR8 | 5′GCAAGTCCCTGGTAGAATTA; | (SEQ ID NO:45) |
| siRNA3, | | |
| TLR8 | 5′GTCGATTCCATTAAGCAATA; | (SEQ ID NO:46) |
| siRNA4, | | |
| TLR9 | 5′GGCAACTGTTATTACAAGA; | (SEQ ID NO:46) |
| siRNA1, | | |
| TLR9 | 5′GCCCTGCAAATACTAGATGT; | (SEQ ID NO:48) |
| siRNA2, | | |
| TLR9 | 5′GGCGAGTGCCCTGCAAATA. | (SEQ ID NO:49) |
| siRNA3, | | |

Western Blotting and RNA Interference

To evaluate the expression levels of each gene, IRAK4 and MyD88 were cloned into FLAG-tagged pcDNA3 expression vector, and inserted TLR7, 8, 9 into an HA-tagged pcDNA3 expression vector. 293 T cells (1.2×106/well) were seeded on 6-well plates for 4 h, and then transfected with 2 μg of plasmid encoding a target gene plus/minus the corresponding or control siRNA plasmid DNAs (2 μg) using Lipofectamine 2000. 48 h later, the transfected cells were lysed and the samples were separated by SDS-PAGE. After the transfer of protein to a PVDF membrane, the expression of a target protein was probed with either an anti-FLAG antibody (M2) for MyD88 and IRAK4 or an anti-HA antibody for TLR7, 8, or 9, followed by a second horseradish peroxidase-conjugated antibody. Beta-actin served as a control for sample loading and was detected with an anti-actin antibody. Western blots were developed with Chemiluminescent Substrate (KPL, Maryland) and visualized on a Kodak Image Station (Eastman Kodak Co.).

Preparation of Lentiviral Supernatants and Transduction of T Cells

5×106 293T cells were pre-seeded onto 100-mm dishes and transfected with 12 μg siRNA lentiviral DNA, 12 μg VSV-G plasmid DNA and 12 μg packaging viral CMV delta 8.9 plasmid, using Lipofectamine 2000. After the addition of fresh culture medium 8 h later, the cells were cultured for an additional 2-3 days. Viral supernatants were harvested, passed through a 0.45 μM filter and concentrated by ultracentrifugation at 20,000 rpm for 2 h. Virus pellets were resuspended in a small volume of medium, and viral titers were determined by infecting 293T cells with serially diluted doses of virus. For transduction of T cells, T cells (2×106 ) were first activated by OKT3 (2 ug/ml)-coated plates and then were mixed with the concentrated lentiviral supernatant with a multiplicity of infection (MOI) of 10-15 in a total volume of 0.5 ml T cell medium containing 8 ug/ml polybrene (Sigma), and then span at 1000×g for 1 h at room temperature. After 16 h of incubation, 0.5 ml of T cell medium was added to each well. Forty-eight hours later, the T cells were transduced again with the same concentrated viral supernatants. Transduction efficiency was analyzed at 3 or 4 days post-transduction, and the cells were sorted into GFP⁺and GFP⁻cells with a FACS ARIA sorter. The sorted cells (GFP⁺and GFP⁻) and untransduced Treg cells were then used to determine their reversibility by Poly-G10 in functional proliferation assays.

TLR Ligand Assays

Näive CD4 T+cells were purified from PBMCs by using microbeads (Miltenvi Biotec). Näive CD4⁺ T cells (105/well) were cultured with regulatory T cells at a ratio of 10:1 in OKT3 (2 ug/ml)-coated, U bottomed 96-well plates containing the following ligands: LPS (100 ng/ml), imiquimod (10 μg/ml), loxoribine (500 μM), poly(I:C) (25 μg/ml), ssRNA40/LyoVec (3 μg/ml), ssRNA33/LyoVec (3 μg/ml), pam3CSK4 (200 ng/ml) and flagellin (10 μg/ml) were purchased from Invivogene (San Diego, Calif.), while CpG-A (3 μg/ml), CpG-B (3 μg/ml) and Poly-G oligonucleotides (3 μg/ml) were synthesized in Integrated DNA Technologies, Inc (Coralville, Iowa). Functional assays were identical to those described above.

Rag 1 ^{-/-} and Rag 2 ^{-/-} γC ^{-/-} mouse experiments

Human 586mel tumor cells (5×106) in 100 μl of buffered saline were subcutaneously injected into Rag1^{-/-} mice (lacking T and B cells) or Rag 2 ^{-/-} γC ^{-/-} mice (lacking T, B and NK cells) on day 0. Tumor-specific CD8⁺ TIL586 cells (2.5×107), which recognize and kill 586mel cells, were injected intravenously on day 3 with or without Treg102 cells (3×106) pretreated with Poly-G10 or Poly-T10. Tumor size was measured with calipers every 2-3 days. Tumor volume was calculated on the basis of two-dimensioned measurements. Tumor growth curves were compared with the Wilcoxon rank-sum test.

Reversal of the suppressive function of CD4⁺ Treg cells by CpG-A.

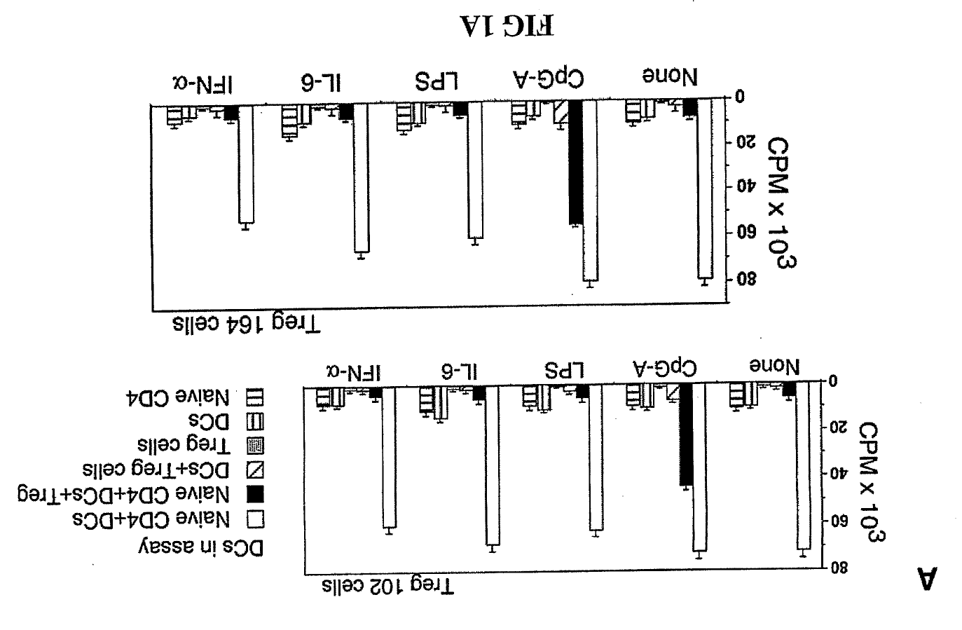

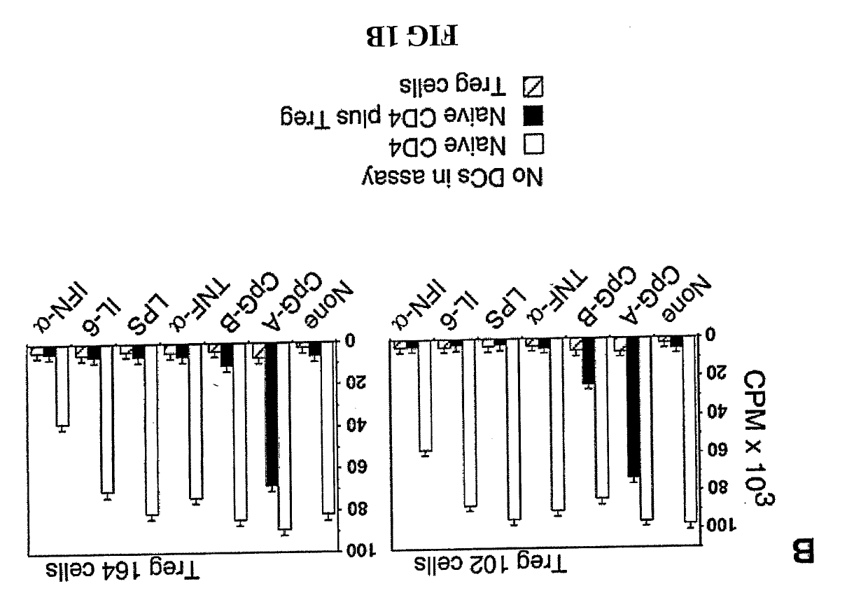

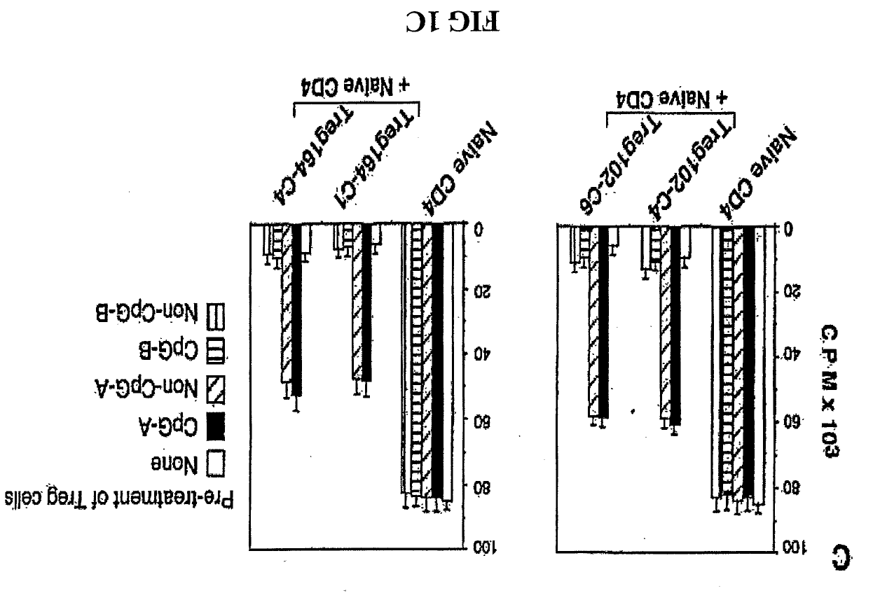

Two CD4⁺ Treg cell lines, designated Treg102 and Treg164, were generated with a panel of Treg cell clones derived from TIL102 and TIL164 cells. As expected, both lines effectively suppressed CD4⁺ CD25⁻ naive T cell proliferation in medium containing soluble anti-CD3 antibody (100 ng/ml) and human DCs, which were generated from peripheral blood mononuclear cell (PBMC)-derived monocytes in medium containing GM-CSF (800 ng/ml) and IL-4 (500 ng/ml) after 7 days of culture (FIG. 1A). A functional proliferation assay was employed to examine whether CpG (a TLR9 ligand), LPS (a TLR4 ligand) or the cytokines IL-6 or IFN-α could regulate the suppressive activity of antigen specific human CD4⁺ Treg cells in the presence of DCs. Activation of DCs with a representative CpG-A oligonucleotide, SEQ ID NO: 1, (G* GGGGACGATCGTCG* G*G*G*G*G, where * stands for phosphorothioate linkages, also called type-D CpG oligonucleotides), reversed Treg cell-mediated suppression, restoring the proliferation of naive CD4⁺ T cells to 50-80% of normal levels (FIG. 1A). This effect was also observed for both the Treg102 and Treg164 cell lines even at a high (1:1) ratio of Treg cells to responder CD4⁺ T cells. By contrast, treatment with LPS, IL-6 or IFN-α did not restore the proliferative ability of naive CD4⁺ T cells in the presence of CD4⁺ Treg cells (FIG. 1A). Interestingly, these TLR ligands and cytokines, either alone or in combination, did not appreciably affect the proliferation of naive CD4⁺ T cells, DCs, Treg cells by themselves, Treg cells plus DCs or naive T cells plus DCs. (FIG. 1A).

Näive CD4⁺ T cell proliferation experiments were performed similar to those in FIG. 1A, but in the absence of DCs. The purified naive CD4⁺ T cells vigorously proliferated in plates coated with anti-CD3 antibody (2μg/ml), and such proliferation could be effectively suppressed in the presence of CD4⁺ Treg cells. Surprisingly, CpG-A (SEQ ID NO: 1) reversed Treg cell-mediated suppression even better in the absence of DCs and restored the proliferation of naive CD4⁺ T cells to near normal levels, while it lacked any effect on the proliferation of naive CD4⁺ T cells or Treg cells alone (FIG. 1B). The reversal effect of CpG-A (SEQ ID NO: 1) contrasts with the failure of CpG-B (SEQ ID NO: 2), LPS, TNF-α, IL-6 or IFN-α to block the suppressive activity of CD4⁺ Treg cells against naive CD4⁺ T cells (FIG. 1B). This data shows there is no requirement for DCs to reverse the suppressive function of Treg cells on naive T cell proliferation.

To exclude the possibility that the purified naive CD4⁺ T cells might have been contaminated with a small number of monocytes or DCs, cultured CD4⁺ Treg cells (100% purity) were pretreated with CpG-A (SEQ ID NO: 1) for 3 days. After three washes, the pretreated CD4⁺ Treg cells were mixed with naive CD4⁺ T cells in anti-CD3 antibody-coated plates to evaluate their ability to suppress the proliferation of naive CD4⁺ T cells. As shown in FIG. 1C, the CpG-A (SEQ ID NO: 1) pretreatment reversed the suppressive effect of the CD4⁺ Treg cell lines.

To determine whether the CpG motif in CpG A confers the ability to reverse the suppressive function of CD4⁺ Treg cells, CD4⁺ Treg cell lines were pretreated for 3 days with CpG-A (SEQ ID NO: 1), non-CpG-A (CG changed to GC in CpG-A; SEQ ID NO: 3), CpG B (SEQ ID NO: 2) or non-CpG-B (CG changed to GC in CpG-B; SEQ ID NO: 4), and used them in proliferation assays following extensive washes. Pretreatment with either CpG-A (SEQ ID NO: 1) or non-CpG-A (SEQ ID NO: 3) reversed the suppressive activities of CD4⁺ Treg cells equally well. In contrast, CpG-B (SEQ ID NO: 2) and non-CpG-B (SEQ ID NO: 4) lacked any discernible effect (FIG. 1C; Data are presented as means±SD and represent results from three independent experiments.). These results indicate that CpG-A-mediated regulation of Treg cell suppressive activity does not depend on the CpG motif.

Identification of sequence elements in CpG-A responsible for the direct reversal of the suppressive function of CD4⁺ Treg cells.

To further define the CpG-A (SEQ ID NO: 1) sequences responsible for the observed reversal effect, CpG-NG (SEQ ID NO: 5) and Poly-G10 (SEQ ID NO: 6), were tested for their ability to reverse the suppressive function of Treg cells. Naive CD4⁺ T cells were mixed with CD4⁺ Treg cells in anti-CD3 antibody-coated 96-wells with 200 μl of medium containing CpG-NG (SEQ ID NO: 5), CpG-A (SEQ ID NO: 1) or Poly-G10 (SEQ ID NO: 6). The proliferation assay was conducted as described in FIG. 1. As shown in FIG. 2A, Treg suppression reversal was lost in CpG-NG (SEQ ID NO: 5) treated cells, but was retained or even enhanced in Poly-G10 (SEQ ID NO: 6). In contrast, Poly-A10 (SEQ ID NO: 7), Poly-C10 (SEQ ID NO: 8) and Poly-T10 (SEQ ID NO: 9), all with the same protected phosphorothioate backbone, lacked the ability to reverse the suppressive activity of Treg cells (FIG. 2B). For the experiments shown in FIG. 2B, naive CD4⁺ T cells were mixed with CD4⁺ Treg cells in anti-CD3 antibody-coated 96-wells with 200 μl of medium containing Poly-A10 (SEQ ID NO: 7), Poly-T10 (SEQ ID NO: 9) or Poly-C10 (SEQ ID NO: 8), while the Poly-G10 (SEQ ID NO: 6) oligonucleotides served as a positive control.

A series of oligonucleotides with decreasing numbers of guanosine nucleosides was constructed to determine the minimal number required for the reversal effect. For the experiments in FIG. 2C, CD4⁺ T cells were mixed with CD4⁺ Treg cells in anti-CD3 antibody-coated 96-wells containing medium with protected phosphorothioate linked guanosine nucleoside containing oligonucleotides of various lengths, while the unprotected five guanosines (SEQ ID NO: 10) served as a control. The ability of these single stranded oligonucleotides to reverse the suppressive function of Treg cells gradually increased as the number of guanosines in the oligonucleotides decreased (FIG. 2C), showing that shorter oligonucleotides bind more readily to TLR8. However, a stretch of guanosines (G5) (SEQ ID NO: 10) with a regular phosphodiester backbone failed to reverse the suppressive activity of Treg cells, most likely because of rapid degradation by nucleases. A4G1 (SEQ ID NO: 11), T4G1 (SEQ ID NO: 12), and C4G1 (SEQ ID NO: 13) were also tested and found to reverse the suppressive activity of Treg cells against naive T cells (FIG. 2D), indicating that a single guanosine plus other nucleotides with protected phosphorothioate linkages is sufficient to reverse Treg cell function.

FIG. 2E: Both CD4⁺ CD25⁺ and CD25⁻ T cells were sorted after staining of freshly isolated human CD4⁺ T cells with anti-CD4 and anti-CD25 antibodies. The purity of the sorted T cells was determined by FACS. Data are presented as means±SD and represent results from two independent experiments. The data presented in FIG. 2E shows the ability of an effective Poly-G5 (SEQ ID NO: 14) construct to reverse the suppressive function of naturally occurring CD4⁺ CD25⁺ Treg cells. CD4⁺ T cells from fresh human PBMCs were stained with anti-CD4 and anti-CD25 antibodies, and sorted into CD4⁺ CD25⁺ and CD4⁺ CD25⁻ T cell populations. The purity of the sorted cell populations is shown to the left in FIG. 2E. The sorted CD4⁺ CD25⁺ Treg cells clearly suppressed the proliferation of CD4⁺ CD25⁻ T cells, but their suppressive activity was entirely reversed by treatment with the Poly-G5 (SEQ ID NO: 14) oligonucleotides (FIG. 2E). Taken together, these results show that short oligonucleotides containing two guanosines or AG, TG and CG with phosphorothioate linkages are more effective than longer ones in reversing the suppressive function of either antigen-specific or naturally occurring Treg cells.

MyD88-IRAK4 pathway is required for reversing the suppressive function of Treg cells.

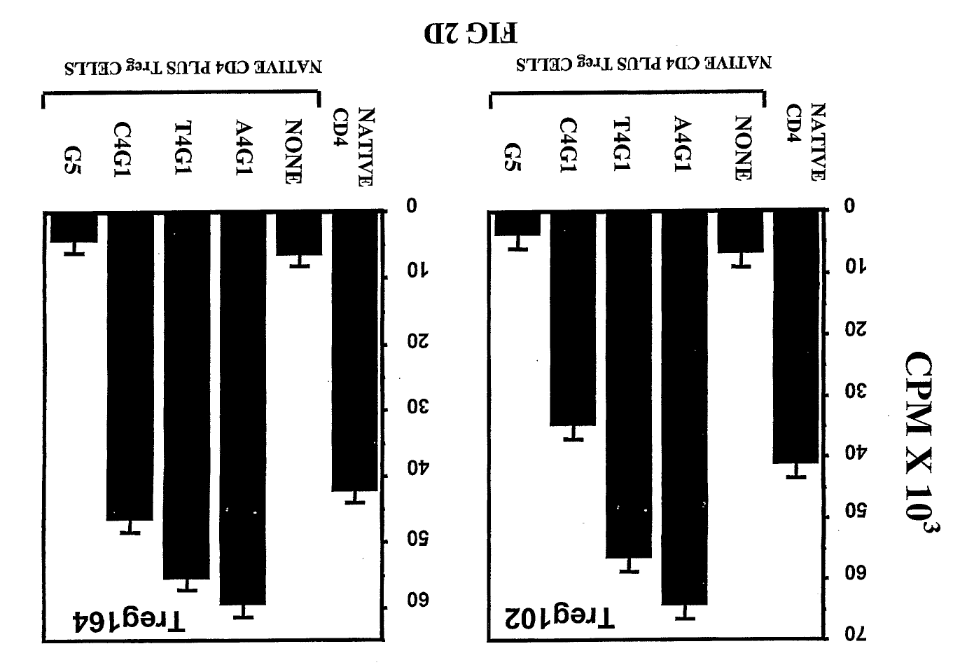

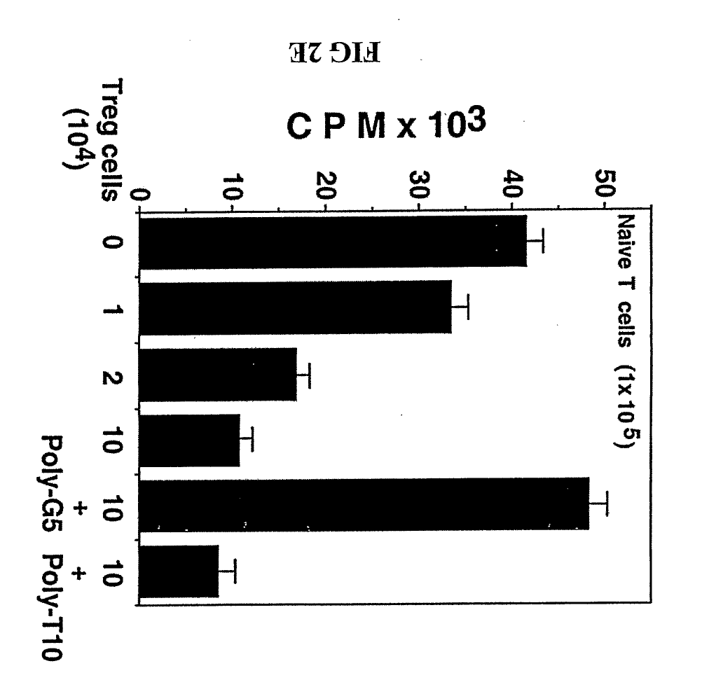

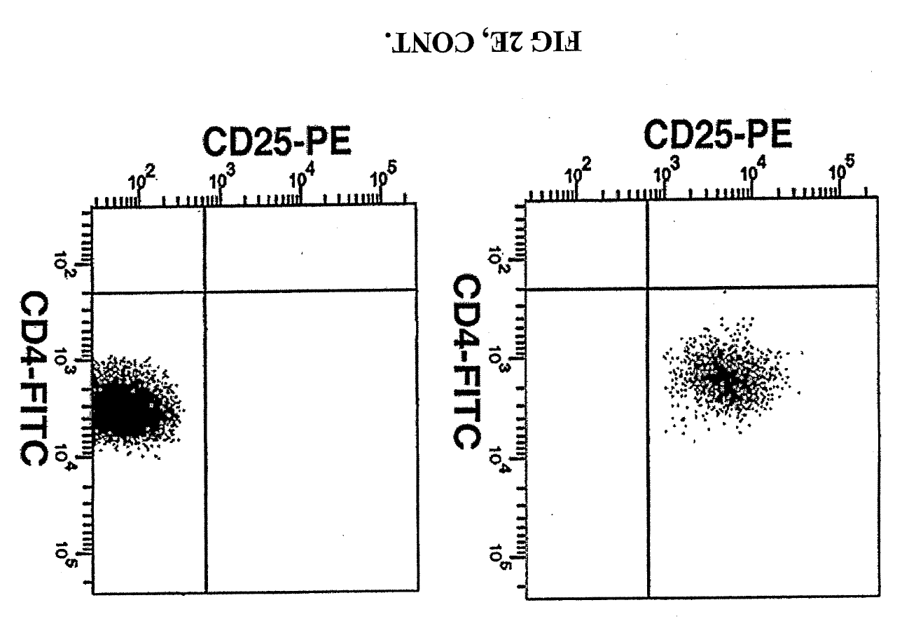

The current model of TLR signaling pathways predicts that TLR1, 2, 5, 6, 7, 8, and 9 use MyD88 as their sole receptor-proximal adaptor to transduce signals, TLR3 uses interferon-regulated factor 3 (IRF3) for the production of IFN-α in response to pathogen recognition, while TLR4 is linked to both MyD88-dependent and MyD88 independent pathways. Since MyD88 is important for the signaling activities of most TLRs, it stood to reason that its dependent pathway, in which IRKA4 transduces signals from MyD88 to TRAF6 and other downstream molecules, would likely be involved in TLR signaling initiated by guanosine-containing oligonucleotides. Four or five eGFP-expressing lentiviral constructs containing U6-driven siRNAs were made for each gene and were tested their abilities to specifically knock down their respective target proteins. Representative data for functional IRAK4 and MyD88 siRNA constructs are shown in FIG. 3A. In FIG. 3A, the efficiency of knock down by siRNA was determined by Western blot analysis. Both IRAK4 and MyD88 were tagged with a FLAG epitope, and detected by anti-FLAG antibody (M2). 293T cells transfected with either FLAG-IRAK4 or FLAG-MyD88 served as positive controls, while nontranseffected 293T cells were negative controls. The amount of β-actin in each lane served as loading controls for the samples. By Western blot analysis, IRAK4 and MyD88 were specifically knocked down by the corresponding siRNAs, while the control TLR9 siRNA did not affect the expression of either protein. Treg102 cells were next transduced with an IRAK4 siRNAl lentivirus. After 3 days in culture, they were sorted by FACS into transduced (GFP⁺) and untransduced (GFP⁻ control) Treg cell populations (FIG. 3B). The purity of GFP⁺ and GFP⁻ cells was confirmed by FACS analysis. The GFP⁺ Treg cells possessed the same reversible suppressive function as the untransduced parental cells (FIG. 3C). Although the transduced (GFP⁺) Treg cells could suppress the proliferation of naive CD4⁺ T cells, this activity could not be reversed with Poly-G10 (SEQ ID NO: 6) oligonucleotides, suggesting that IRAK4 is required for the direct reversal of Treg cell suppressive function. This notion was further supported by experiments in which Treg102 cells were transduced with MyD88 siRNAs. The transduced (GFP⁺) Treg102 cells completely lost their ability to respond to treatment with Poly-G10 (SEQ ID NO: 6), even though they could still suppress naive CD4⁺ T cell proliferation (FIG. 3B). Neither the suppressive activity nor the reversibility of the suppression was affected when Treg cells were transduced with a control siRNA virus (FIG. 3C). For FIG. 3C, uninfected parental Treg108 cells and Poly-T10 oligonucleotides served as controls. Data are presented as means±SD and represent results from three independent experiments. Similar results were obtained with Treg164 cell lines.

This data demonstrates that direct reversal of the suppressive activity of Treg cells by guanosine containing oligonucleotides relies on TLR signal transduction through the MyD88-IRAK4 pathway. Hence, the receptors for such ligands must be limited to those using MyD88-IRAK4 as their sole or primary signaling pathway. A review of the properties of all known TLR ligands indicated that TLR3, 7, 8 and 9 would be the most likely candidates for binding nucleotide-related ligands. Although TLR3 recognizes double-stranded RNA, it relies on a MyD88-IRAK4-independent signaling pathway. TLR7, 8 and 9, which form an evolutionary cluster, are thought to reside in endosomes and to initiate signaling through the MyD88-IRAK4 pathway. TLR9 has been identified as a receptor for CpG oligonucleotides, while TLR7 and 8 function as receptors for synthetic guanosine analogs (loxoribine and imidazoquinoline) and single-stranded RNA. Although some TLRs have been reported to be expressed by mouse CD4⁺ CD25⁺ Treg cells, their expression by human CD4⁺ Treg cells are first demonstrated by the data herein.

TLR8 is the receptor responsible for guanosine oligonucleotide-induced reversal of the suppressive function of Treg cells.

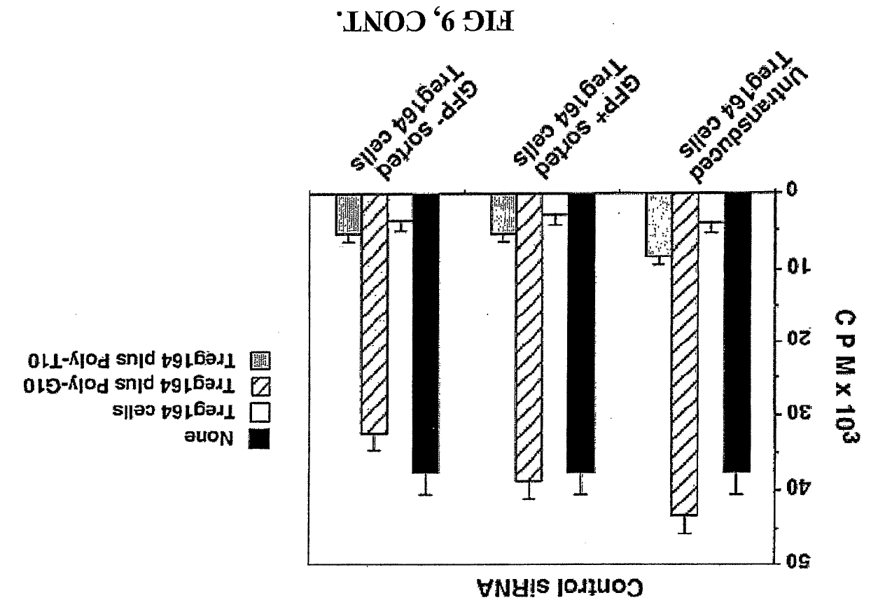

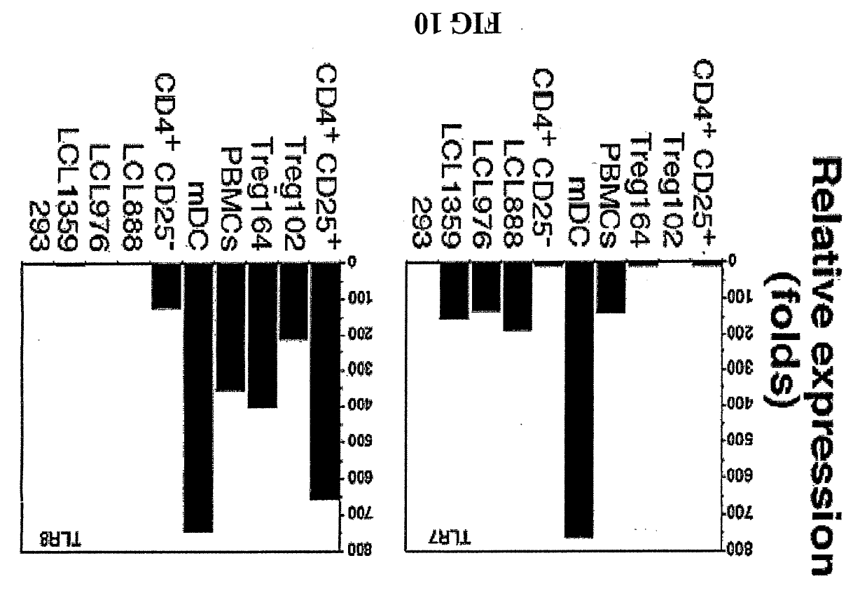

Reverse-transcription PCR revealed that neither TLR7 nor TLR9 was expressed in antigen specific Treg cell lines, CD4⁺ CD25⁺ Treg cells or CD4⁺ CD25⁻ T cells, although TLR7 was highly expressed in DC, and EBV-transformed B cells (FIG. 10). By contrast, TLR8 was consistently expressed by naturally occurring CD4⁺ CD25⁺ Treg cells, antigen-specific Treg cell lines, PBMCs, monocyte-derived DCs (mDCs) as well as CD4⁺ CD25⁻ T cells, but was not detectable in EBV-transformed B cells or 293 cells (FIG. 10). Similar results were obtained with real-time PCR for TLR7 and 8. TLR7 was negative in all T cells; TLR8 was highly expressed in CD4⁺ Treg cells, but weakly expressed in CD4⁺ CD25⁻T cells (FIG. 10; TLR7 and 8 expression determined by real-time PCR analysis of cDNA, from each sample using primers and internal fluorescent probes for TLR7, 8 or HPRT (hypoxanthine-guaninephosphoribosyltransferase). The relative quantity of TLR7 and 8 in each sample was normalized to the relative quantity of HPRT.).

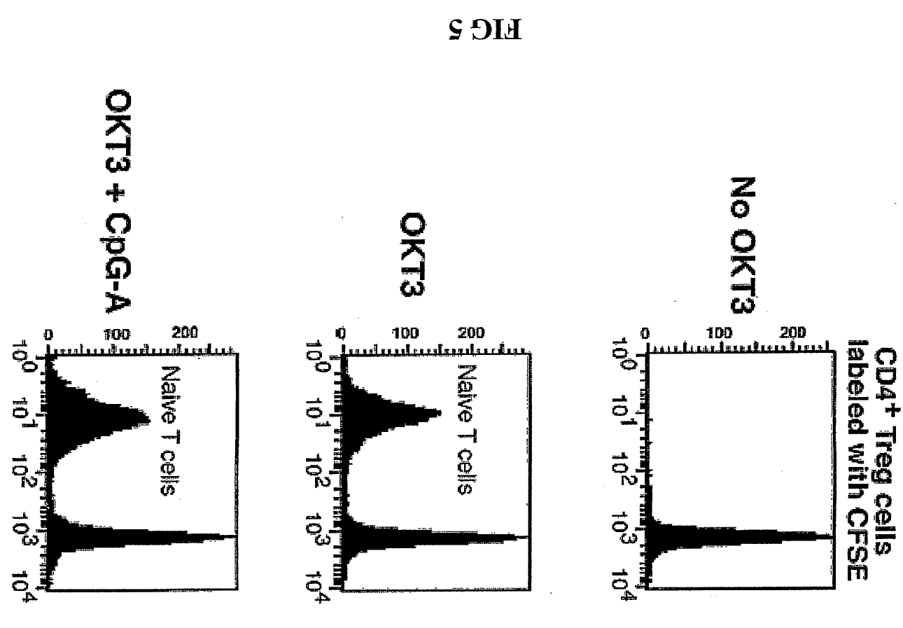

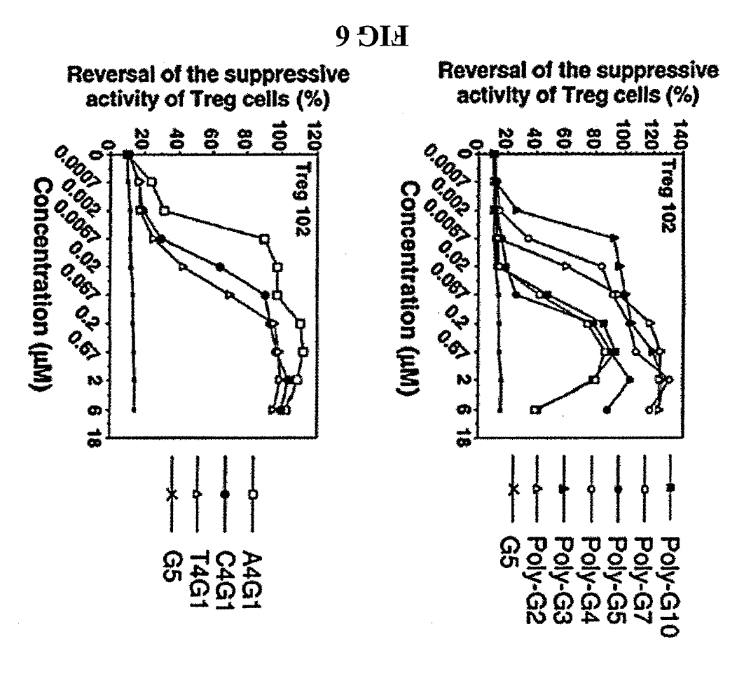

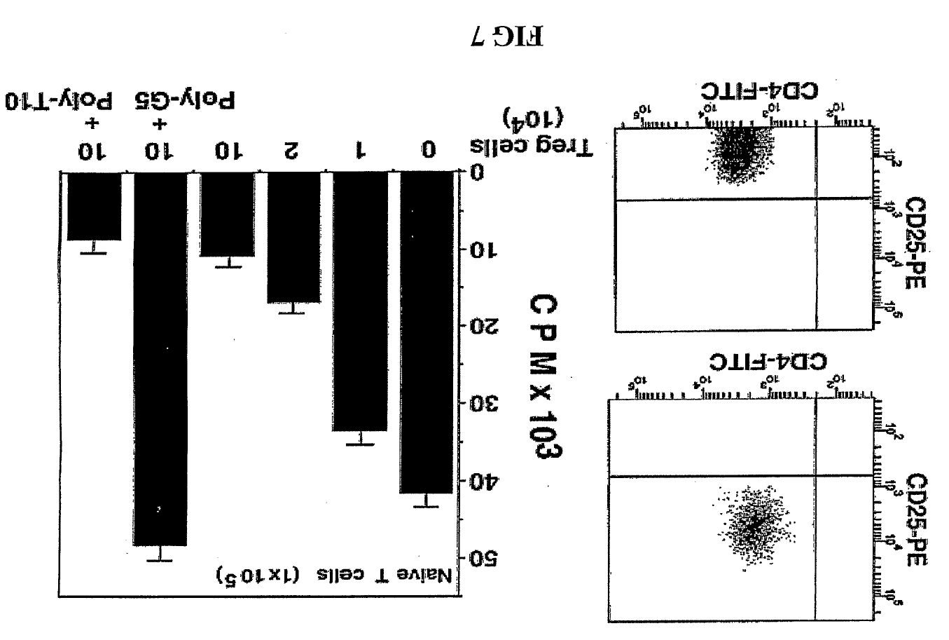

On the basis of the expression pattern of TLR7, 8 and 9 in Treg cells, TLR8 was the likely receptor for guanosine containing oligonucleotides. Several lentiviral siRNA constructs were screened against TLR7, 8 and 9. Representative data for functional knock down of TLR7, 8 and 9 by the corresponding siRNAs are shown in FIG. 8 (Determined by Western blot analysis with an anti-FLAG antibody (M2)). Treg102 cells were infected with TLR8 siRNAl virus, and sorted into transduced (GFP⁺) and untransduced (GFP⁻) Treg populations. Untransduced parental Treg102 cells served as a control for the functional assay. The suppressive function of Treg102 cells transduced with TLR8 siRNA (GFP⁺) could not be reversed by Poly-G10 (SEQ ID NO: 6), in contrast to untransduced (GFP⁻) Treg102 cells, whose suppressive activity was reversed as readily as the parental Treg102 cells (FIG. 3C & 4B); Treg 102 cells were infected with TLR7 siRNA 1, TLR8 siRNA 1 or TLR9 siRNA 1, and 3 days later were sorted into transduced (GFP⁺) and untransduced (GFP⁻) cell populations, which were tested in a functional assay in the presence of Poly-G10 (SEQ ID NO: 6) or Poly-T10 (SEQ ID NO: 9)). Treg cells transduced with TLR7 siRNA or TLR9 siRNA (GFP⁺) retained the same reversible suppressive function as untransduced (GFP⁻) and parental Treg102 cells, suggesting that TLR8, but not TLR7 or TLR9, is indeed the receptor recognizing guanosine containing oligonucleotides and initiating signals through the MyD88-IRAK4 pathway that control the suppressive function of Treg cells.

If the TLR8-MyD88-IRAK4 signaling pathway is necessary and sufficient for direct reversal of the suppressive function of Treg cells, it should be possible to produce that effect with natural ligands for human TLR8. For FIG. 4A-B, naive CD4⁺ T cells were mixed with Treg102, Treg164 or naturally occurring CD4⁺ CD25⁺ Treg cells in anti-CD3 antibody-coated wells in the presence of different TLR ligands. Data are presented as means±SD and represent results from three independent experiments. As shown in FIG. 4A-B, ssRNA40 and ssRNA33, two natural ligand for human TLR8, completely reversed the suppressive function of antigen-specific Treg102 and Treg164 cells, as well as naturally occurring CD4⁺ CD25⁺ Treg cells. Imiquimod, a synthetic ligand for human TLR7 and 8, showed partial reversal of the suppressive function of antigen-specific Treg cells, but little or no effect on naturally occurring CD4⁺ CD25⁺ Treg cells (FIG. 4A-B). Other ligands such as pamsCSK4 for TLR2, poly(I:C) for TLR3, LPS for TLR4, flagellin for TLR5, loxoribine for TLR7, and CpG-B (SEQ ID NO: 2) for TLR9 failed to restore the proliferation of naive CD4⁺ T cells in the presence of Treg cells (FIG. 4A-B).

The data in FIG. 4A-B confirms that TLR8 serves as a receptor for synthetic guanosine containing oligonucleotides and natural ssRNA40 and ssRNA33 ligands derived from HIV-1 virus, and that its activation in endosomes by synthetic DNA or ssRNA ligands triggers a signaling pathway that can reverse the suppressive function of Treg cells. To study the guanosine containing oligonucleotide induced reversal of Treg cell function in vivo, a human tumor model was established by subcutaneously injecting 586mel human tumor cells into Rag1^{-/-} (T and B cell deficient) mice. Rag 1-deficient mice were injected with human 586mel tumor cells on day 0, and then treated with autologous tumor-specific CD8⁺ TIL586 cells alone or CD8⁺ TIL586 cells plus Treg102 cells with or without Poly-G10 (SEQ ID NO: 6) or Poly-T10 (SEQ ID NO: 9) on day 3. Treg cells were pre-activated with OKT3 and washed before adoptive transfer. Tumor volumes were measured and presented as mean±SD (n=6 mice per group). P value was determined between groups as indicated. Results are a representative of three independent experiments. As expected, mice receiving 586mel tumor cells showed progressive tumor growth, while in mice receiving 586mel plus autologous tumor-specific CD8⁺ TIL586 cells, which can kill 586mel cells, tumor growth was inhibited (FIG. 4C). When CD8⁺ TIL586 and Treg102 cells with or without Poly-T10 (a control oligonucleotide; SEQ ID NO: 9) were adoptively transferred into tumor bearing mice, 586mel cells grew faster than in mice receiving 586mel cells alone, suggesting that the Treg102 cells inhibited tumor-specific CD8⁺ T cells and residual host immune cells, thus enhancing tumor growth. By contrast, in mice receiving Poly-G10 (SEQ ID NO: 6), Treg102 and TIL586 cells, tumor growth was not detected during the first 42 days after tumor injection of 586mel cells (FIG. 4C), indicating that guanosine containing oligonucleotide treatment not only reversed the suppressive function of Treg cells, but also dramatically enhanced T cell-mediated antitumor immunity in vivo.

Treg cells were pretreated with Poly-G2 (SEQ ID NO: 15) in different concentrations of chloroquine to examine whether acidification is required for the function of guanosine containing oligonucleotides. After extensive washes, the pretreated Treg cells were used for functional assays. The reversal effect of Poly-G2 (SEQ ID NO: 15) oligonucleotides decreased with increasing concentrations of chloroquine, suggesting that binding and activation of TLR8 in endosomes by either guanosine containing DNA or RNA oligonucleotides is the key step in reversal of Treg suppressive function.

Proliferation and Cytotoxicity of Treg Cells

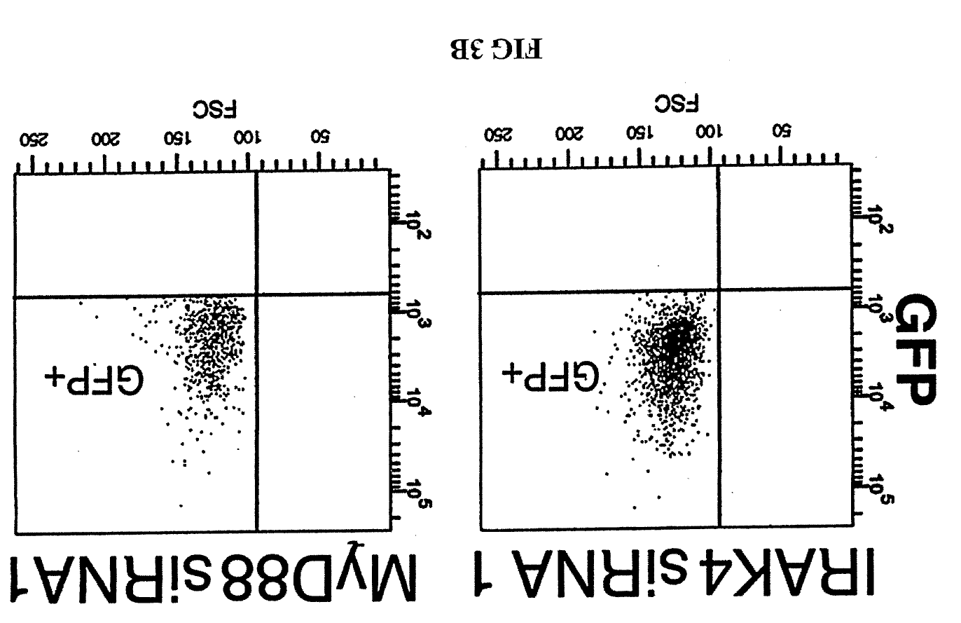

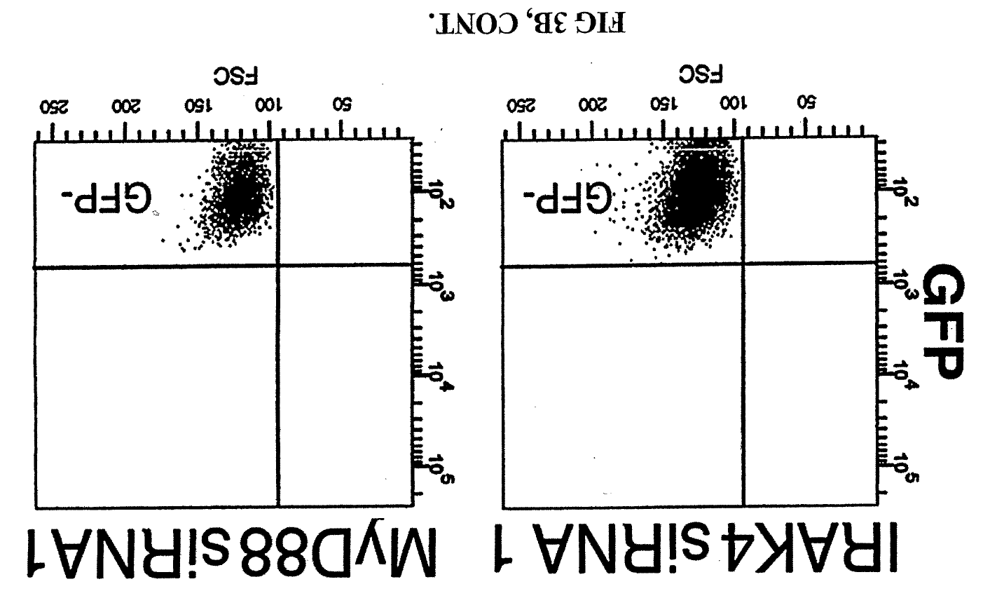

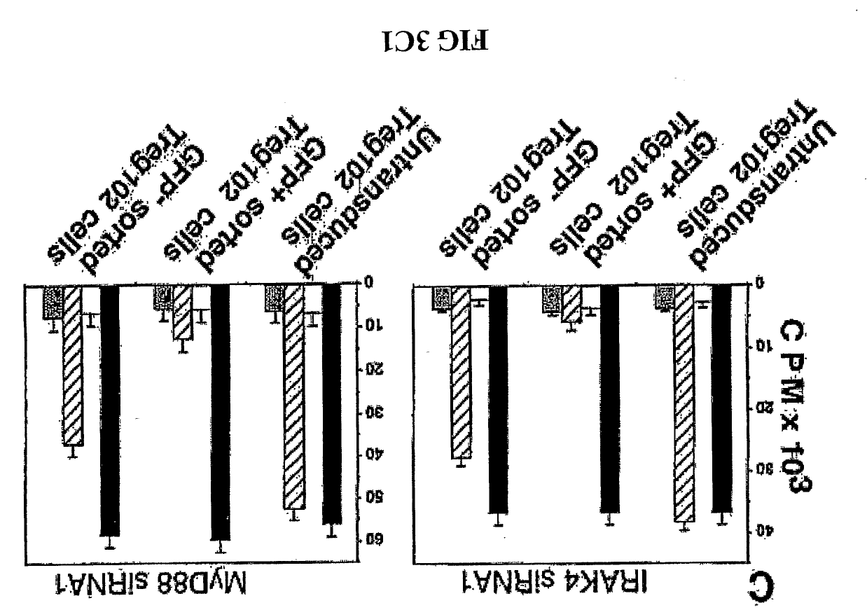

FIG. 5. Treg102 cells (1×107) were labeled with CFSE, cultured in T cell medium (RPMI 1640/10% human serum and 300 IU of IL-2), and divided into three groups: 1) CFSE-labeled Treg cells without OKT3 stimulation (control), 2) CFSE-labeled Treg cells mixed with equal numbers of näive CD4⁺ T cells in the presence of OKT3, and 3) CFSE-labeled Treg cells mixed with equal numbers of näive CD4⁺ T cells in the presence of OKT3 and CpG-A (3 μg/ml). After 3 days of culture, the proliferative profile was determined by FACS analysis. CFSE-labeled Treg cells without OKT3 stimulation were used as a control for gating. Dead cells were excluded by propidium iodide staining. The results indicate that Treg cells do not proliferate in the presence of näive T cells, OKT3, IL-2 and/or CpG-A. The number of Treg cells did not change in the presence or absence of CpG-A, suggesting that Treg cells are not cytotoxic.

Identification and titration of sequence elements in CpG-A responsible for the direct reversal of Treg cell suppressive function.

FIG. 6. A. Dose response of Poly-G oligonucleotides to reversal of Treg cell suppressive function. Näive CD4⁺ T cells were mixed with CD4⁺ Treg cells in anti-CD3 antibody-coated 96-wells containing medium with different concentrations of Poly-Gs of various lengths, while five guanosines with regular phosphodiester bonds (G5) served as a control. Data are presented as the percent reversal of näive CD4⁺ T cell proliferation in the presence of Treg cells, with näive T cell proliferation in the absence of Treg cells taken as 100%.

B. Dose response relationships for the reversibility of Treg cells by A4G1, C4G1 and T4G1 oligonucleotides. Experiments were performed as in panel A.

Purification and suppressive function of CD4⁺ CD25⁺ Treg cells and their functional reversal by Poly-G5.

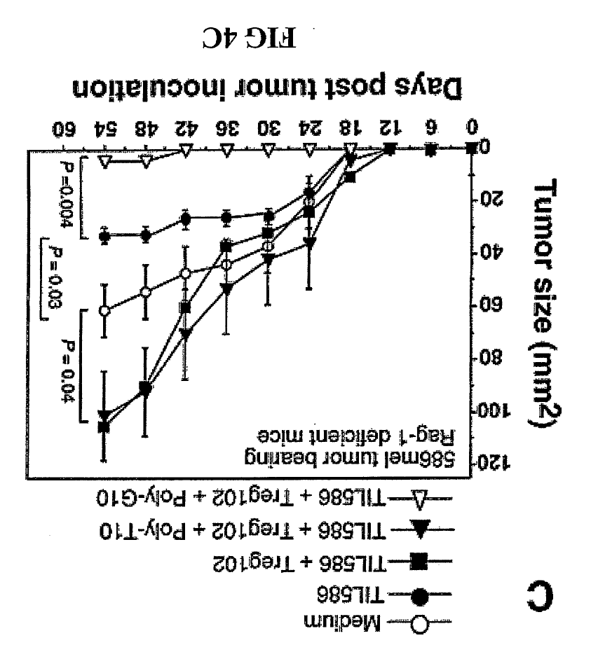

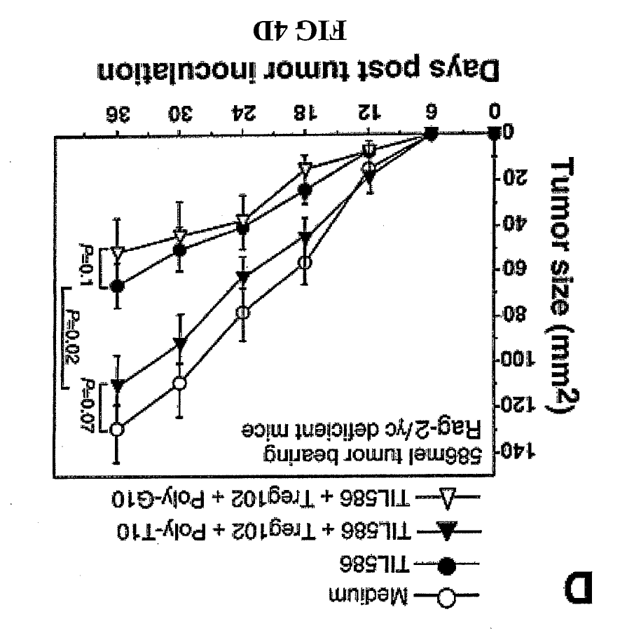

FIG. 7. The purity of the sorted CD4⁺ CD25⁺ Treg cells and CD4⁺ CD25⁻ T cells was determined by FACS. The suppressive function of CD4⁺ CD25⁺ Treg cells was determined by adding different numbers of CD4⁺ CD25⁺ Treg cells (as indicated) to a fixed number of näive T cells. The suppressive function of naturally occurring CD4⁺ CD25⁺ Treg cells could be reversed by Poly-G5, but Poly-T10.

Knockdown of IRAK4. MyD88, TLR7, TLR8 and TLR9 by RNA interference.

FIG. 8. Both IRAK4 and MyD88 were tagged with a FLAG epitope, while TLR7, 8, 9 were cloned into an HA-tagged pcDNA3 expression vector. Expression levels and the efficiency of knockdown for each gene were determined by Western blot analysis using anti-FLAG (M2) or anti-HA antibodies. The amount of β-actin in each lane served as a loading control for the samples.

The MyD88-IRAK4 pathway is required to reverse the suppressive function of Treg164 cells.

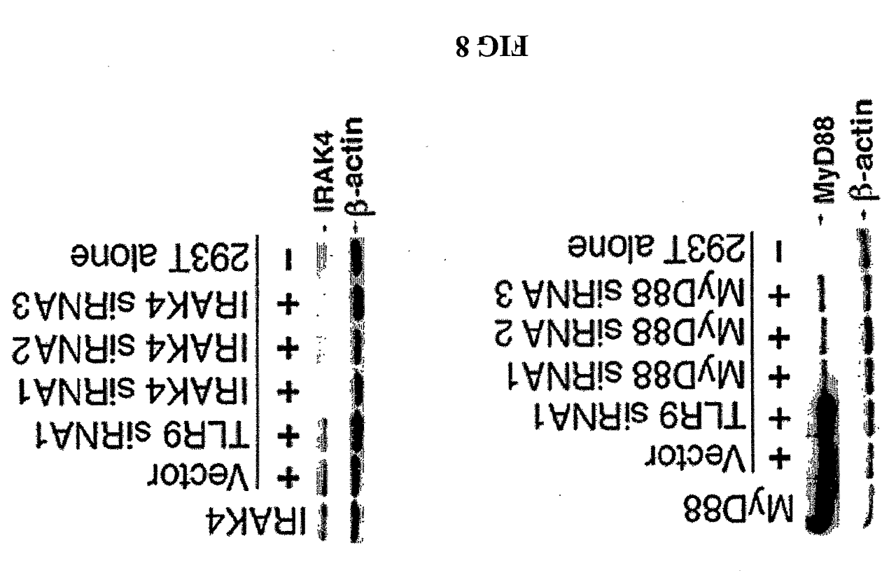

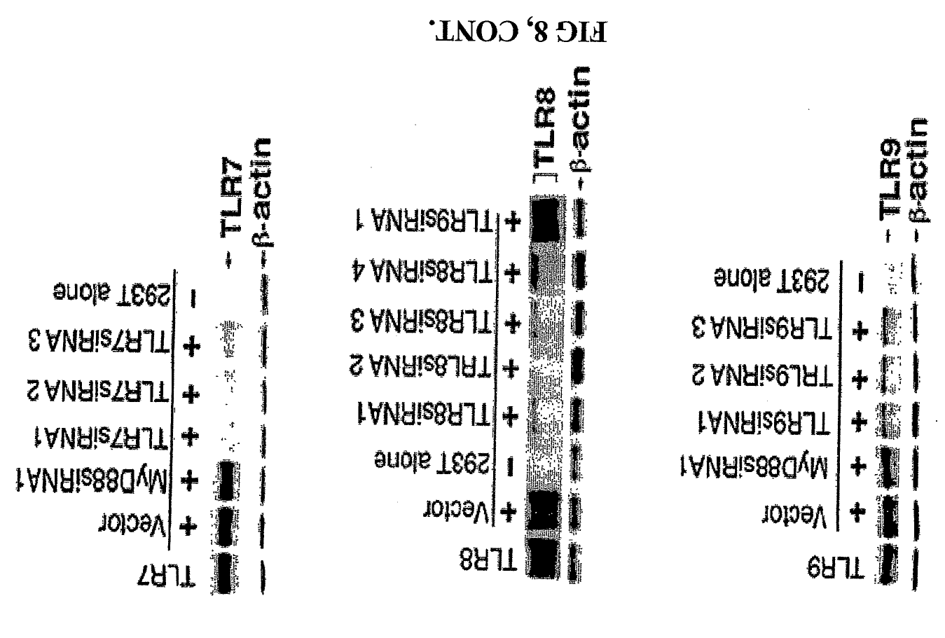

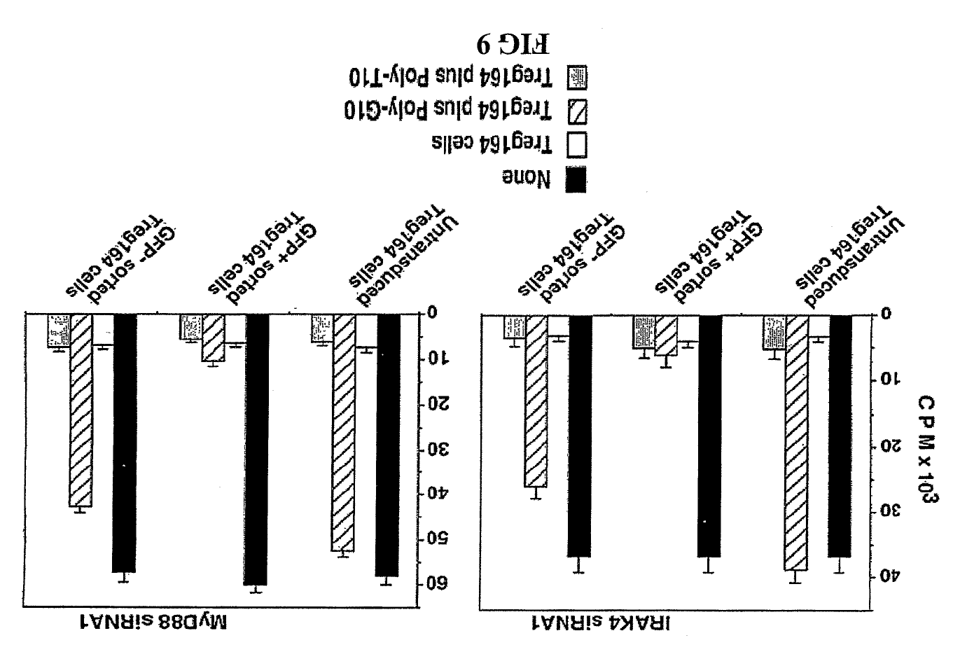

FIG. 9. Evaluation of the reversibility of transduced (GFP⁺) and untransduced (GFP⁺) Treg164 cells by Poly-G10 oligonucleotides. Uninfected parental Treg164 cells and Poly-T10 oligonucleotides served as controls. Both IRAK4 siRNA- and MyD88 siRNA-transduced (GFP⁺) Treg164 cells lost the capacity to reverse their suppressive function in the presence of Poly-G10. By contrast, transduction with control siRNA lacked any effect on Treg cell-mediated suppression of näive CD4⁺ T cell proliferation.

Expression level of TLR7 and 8 in Treg cells determined by real-time PCR analysis in different cell lines with gene-specific primers.

FIG. 10. RNA from 293 cells served as a negative control, and the relative quantity of TLR7 and 8 in each sample was normalized to the relative quantity of HPRT.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

What is claimed is:

1. A method for suppressing the activity of a CD4⁺ T-regulatory cell comprising providing to the cell an effective amount of an oligonucleotide capable of suppressing the activity of the T-regulatory cell, wherein the oligonucleotide is not a Type D CpG oligonucleotide.

2. The method of claim 1, wherein the oligonucleotide is further defined as a non CpG containing oligonucleotide.

3. The method of claim 1, wherein the oligonucleotide comprises between about 4 and about 15 nucleotide residues.

4. The method of claim 1, wherein the oligonucleotide comprises a guanine and a nuclease resistant inter-residue backbone linkage.

5. The method of claims 4, wherein the oligonucleotide further comprises a nuclease sensitive inter-residue backbone linkage.

6. The method of claims 4 or 5, wherein the oligonucleotide comprises a nuclease resistant inter-residue backbone linkage connecting the guanine to an adjacent nucleobase.

7. The method of claim 1, wherein the cell is within a subject.

8. The method of claim 7, wherein the subject is human.

9. The method of claim 8, further comprising providing the human with an immunogenic composition.

10. The method of claim 1, wherein the oligonucleotide is selected from the group consisting of SEQ ID NOs: 6, 11, 12, 13, 14, 15, 16, 17 and 18, or mixtures thereof.

11. An oligonucleotide comprising a guanine and a nuclease resistant inter-residue backbone linkage connecting the guanine to an adjacent nucleobase, wherein the oligonucleotide has 4 to 15 nucleotide residues and wherein an inter-residue backbone linkage is nuclease sensitive.

12. An oligonucleotide selected from the group consisting of SEQ ID NOs: 6, 11, 12, 13, 14, 15, 16, 17 and 18, or mixtures thereof.

13. A method for screening for a compound which inhibits the suppressive function of Treg cells comprising the steps of,
a. exposing a Treg cell to the compound being screened,
b. stimulating the proliferation of a näive T cell,
c. exposing the näive T cell to the Treg cell, and
d. determining the degree of growth of the näive T cell.

14. The method of claim 13, wherein the compound is selected from a library or collection of compounds.

15. The method of claim 13, wherein the degree of growth of the näive T-cell is determined by comparison to the growth of a control näive T-cell.
